(19) United States
(12) Patent Application Publication
(10) Pub. No.: US 2023/0025559 A1
(43) Pub. Date: Jan. 26, 2023

(54) GASTRIC RESTRICTIVE DEVICE

(71) Applicant: Davide LOMANTO, Singapore (SG)

(72) Inventors: Amir SHAKOURI, Singapore (SG); Shahrzad RAYATPISHEH, Singapore (SG); Davide LOMANTO, Singapore (SG); Teng Yong NG, Singapore (SG)

(21) Appl. No.: 17/783,668

(22) PCT Filed: Dec. 9, 2020
(86) PCT No.: PCT/SG2020/050732

§ 371 Date: Jun. 9, 2022

(30) Foreign Application Priority Data
Dec. 10, 2019 (SG) 10201911888S

(51) Int. Cl. A61F 5/00

(52) U.S. Cl. CPC A61F 5/0086; A61F 5/003

(57) ABSTRACT

A gastric restrictive device including two separate elongated rigid members, at least two elongated inflatable paddings respectively along the two elongated rigid members, at least one flexible and extendable hollow tube fluidly interconnecting the at least two elongated inflatable paddings at inner ends thereof, and a two-part interlocking mechanism at outer ends of the two elongated rigid members. The at least one flexible and extendable hollow tube being bendable to fold the two elongated rigid members towards each other into a closed configuration with the two elongated inflatable paddings parallel and opposing each other and with the two-part interlocking mechanism interlockable. The device is attachable to a stomach and the at least two elongated inflatable paddings are inflatable by injecting fluid through the at least one flexible and extendable hollow tube for partially restricting the stomach without damaging the stomach tissue and with maintaining normal physiological functions of the stomach.

### CROSS-REFERENCE TO RELATED APPLICATIONS

The present application claims the benefit of the Singapore patent application No. 10201911888S filed on 10 Dec. 2019, the entire contents of which are incorporated herein by reference for all purposes.

### TECHNICAL FIELD

Various embodiments generally relate to a gastric restrictive device. In particular, various embodiments generally relate to a gastric restrictive device for a reversible bariatric surgery that restricts an effective size of a stomach without removing any portions of the stomach.

### BACKGROUND

Bariatric surgery is a surgical approach for the treatment of obesity. It is typically the last line of intervention for patients with morbid obesity. In these patients, bariatric surgery has expanded longevity and decreased long-term mortality, morbidity and health care use.

Among bariatric surgeries, sleeve gastrectomy has become very popular over the last decade, and conversely gastric banding procedures have dramatically declined. This appears to be due to sleeve gastrectomy being more effective in weight loss with less probability of reoperation and/or complication as compared to gastric banding. However, sleeve gastrectomy is irreversible, involving a surgical removal of a large portion of the stomach and requiring an invasive surgery and a longer recovery time.

Accordingly, there is a need to provide a more effective and balanced solution to address some of the issues above.

### SUMMARY

According to various embodiments, there is provided a gastric restrictive device. The gastric restrictive device may include two separate elongated rigid members. The gastric restrictive device may include a cushioning arrangement. The cushioning arrangement may include at least one first elongated inflatable padding lined longitudinally along a first elongated rigid member of the two separate elongated rigid members. The cushioning arrangement may include at least one second elongated inflatable padding lined longitudinally along a second elongated rigid member of the two separate elongated rigid members. The cushioning arrangement may include at least one flexible and extendable hollow tube fluidly interconnecting the at least one first elongated inflatable padding and the at least one second elongated inflatable padding. A first end of the at least one flexible and extendable hollow tube may be connected to an end of the at least one first elongated inflatable padding corresponding to a first longitudinal end of the first elongated rigid member, and a second end of the at least one flexible and extendable hollow tube may be connected to an end of the at least one second elongated inflatable padding corresponding to a first longitudinal end of the second elongated rigid member. The gastric restrictive device may include an interlocking arrangement. The interlocking arrangement may include a first interlocking element at a second longitudinal end of the first elongated rigid member, and a second interlocking element at a second longitudinal end of the second elongated rigid member. The at least one flexible and extendable hollow tube of the cushioning arrangement may be bendable to fold the two separate elongated rigid members towards each other from a non-closed configuration, whereby the first interlocking element and the second interlocking element are spaced apart from each other in a non-engageable disposition, into a closed configuration with the at least one first elongated inflatable padding and the at least one second elongated padding parallel and opposing each other and with the first interlocking element and the second interlocking element engageable with each other to interlock in the closed configuration.

According to various embodiments, the gastric restrictive device may further include a securing arrangement. The securing arrangement may have a stomach fundus securing component along the first elongated rigid member or the second elongated rigid member, and at least one stomach body securing component along the at least one flexible and extendable hollow tube of the cushioning arrangement and interposed between the first end and the second end of the at least one flexible and extendable hollow tube.

### BRIEF DESCRIPTION OF THE DRAWINGS

In the drawings, like reference characters generally refer to the same parts throughout the different views. The drawings are not necessarily to scale, emphasis instead being generally placed upon illustrating the principles of the invention. In the following description, various embodiments are described with reference to the following drawings, in which:

FIG. 1A shows a schematic diagram of a gastric restrictive device according to various embodiments;

FIG. 1B shows a schematic diagram of the gastric restrictive device of FIG. 1A in a closed or folded configuration according to various embodiments;

FIG. 1C shows a schematic diagram of the gastric restrictive device of FIG. 1B in the closed configuration with the at least one flexible and extendable hollow tube being extended according to various embodiments;

FIG. 2A shows a gastric restrictive device, as an example of the gastric restrictive device of FIG. 1A to FIG. 1C, according to various embodiments;

FIG. 2B shows a A-A cross-section of the gastric restrictive device of FIG. 2A according to various embodiments;

FIG. 2C shows a B-B cross-section of the gastric restrictive device of FIG. 2A according to various embodiments;

FIG. 2D shows an enlarged view of a circled portion C of FIG. 2C according to various embodiments;

FIG. 2E shows an enlarged view of a circled portion D of FIG. 2A according to various embodiments;

FIG. 2F shows an enlarged view of a circled portion E of FIG. 2A according to various embodiments;

FIG. 2G shows an enlarged view of a circled portion F of FIG. 2A according to various embodiments;

FIG. 2H shows the gastric restrictive device of FIG. 2A in a closed configuration according to various embodiments;

FIG. 2I shows the gastric restrictive device of FIG. 2A clamped or clipped or fitted to a stomach according to various embodiments;

FIG. 3 shows an example illustration of the contact and sealing pressure obtained from simulation studies according to various embodiments; and

FIG. 4 shows an example illustration of the rise of sealing pressure with injection of water-based solution obtained from the simulation studies according to various embodiments.

### DETAILED DESCRIPTION

Embodiments described below in the context of the apparatus are analogously valid for the respective methods, and vice versa. Furthermore, it will be understood that the embodiments described below may be combined. For example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments generally relate to a gastric restrictive device. In particular, various embodiments generally relate to a gastric restrictive device for a reversible bariatric surgery that restricts an effective size of a stomach without removing any portions of the stomach. According to various embodiments, the gastric restrictive device may include a gastric clamp or a gastric clip or a gastric band or a gastroplasty device.

Various embodiments seek to provide a solution based on gastric clamping/clipping/banding as a less invasive approach for bariatric surgery without altering the anatomy of stomach or any gastrointestinal organ. Accordingly, a surgery involving the various embodiments may be fully reversible. Various embodiments may also be configured based on the unique anatomy and physiology of the stomach. For example, various embodiments may be configured to address the anatomy of the stomach, whereby different parts of the stomach have different muscle thickness for their intended purpose in digestion, as well as the physiology of the stomach such as the peristalsis movement of the stomach. Various embodiments also seek to provide a solution that, in addition to saving costs and adding years to life, may additionally provide a better quality of life as compared to conventional bariatric surgery.

Various embodiments seek to provide a gastric restrictive device for clamping the stomach without tissue damage and maintaining normal physiological functions of the stomach (mainly the peristalsis movements). Various embodiments seek to provide a gastric restrictive device to mimic sleeve gastrectomy without cutting the stomach or changing the anatomy. Accordingly, the gastric restrictive device may be vertically clamped or clipped or fitted to the stomach in a manner so as to reduce the effective size of the stomach into a sleeve or a tube similar to that of the conventional sleeve gastrectomy but with the stomach being intact so as to be reversible. According to various embodiments, the device may have the advantage of being highly compatible with both the physiology and anatomy of the stomach. Thus, the device of the various embodiments may also overcome the side effects of conventional gastric banding.

According to various embodiments, the gastric restrictive device may be implanted through laparoscopic surgery (single port or natural orifice) to the exterior of the stomach in order to restrict the food intake by preventing the expansion of the fundus part of the stomach. The gastric restrictive device of the various embodiments may lead to semi-isolation of the stomach fundus which may lead to less food intake (significant weight loss) and potentially hormonal changes including an effect on hunger/satiety as well as metabolic benefits for patients, for example patients with metabolic syndrome and diabetes type H.

Various embodiments seek to provide a gastric restrictive device including two separate elongated rigid members, at least two elongated inflatable paddings respectively along the two elongated rigid members, at least one flexible and extendable hollow tube fluidly interconnecting the at least two elongated inflatable paddings at inner ends thereof, and a two-part interlocking mechanism at outer ends of the two elongated rigid members. The at least one flexible and extendable hollow tube may be bendable to fold the two elongated rigid members towards each other into a closed configuration with the two elongated inflatable paddings parallel and opposing each other and with the two-part interlocking mechanism interlockable. The device may be attachable to a stomach and the at least two elongated inflatable paddings may be inflatable by injecting fluid through the at least one flexible and extendable hollow tube for partially restricting the stomach without damaging the stomach tissue and with maintaining normal physiological functions of the stomach.

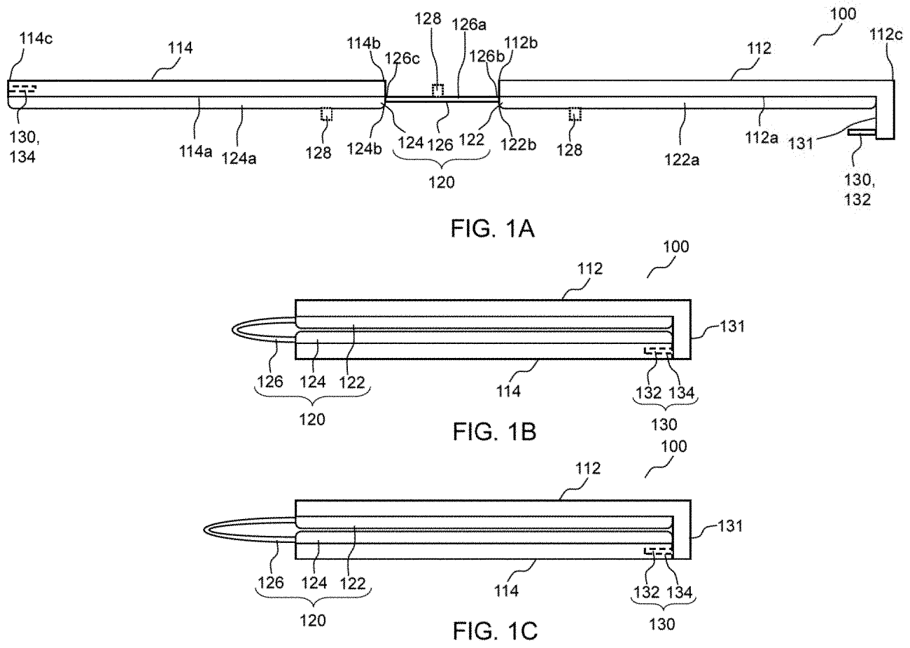

FIG. 1A shows a schematic diagram of a gastric restrictive device 100 according to various embodiments. FIG. 1B shows a schematic diagram of the gastric restrictive device 100 of FIG. 1A in a closed or folded configuration according to various embodiments. The schematic diagrams of the gastric restrictive device 100 are not drawn to scale and are not provided to limit the shape and/or configuration of the various components. Rather, the schematic diagrams of the gastric restrictive device 100 are provided to depict the significant components of the gastric restrictive device 100, with some details exaggerated or introduced, to facilitate the understanding of the gastric restrictive device 100. According to various embodiments, the gastric restrictive device 100 of FIG. 1A may be folded into the closed or folded configuration of FIG. 1B for clamping or clipping or banding of the stomach. According to various embodiments, the gastric restrictive device 100 may be a gastric clamp or a gastric clip or a gastric band or a gastroplasty device. According to various embodiments, the gastric restrictive device 100 in the closed or folded configuration may be configured to restrict the effective size of the stomach without permanently removing any portions of the stomach or permanently changing the anatomy of the stomach.

According to various embodiments, the gastric restrictive device 100 may include two separate elongated rigid members 112, 114. Each of the two separate elongated rigid members 112, 114 may respectively be a solid structure which may be stiff and inflexible.

According to various embodiments, each of the two separate elongated rigid members 112, 114 may be in the form of, including but not limited to, a bar, a rod, a pole, a stripe, a rib, or a stick of solid material. Accordingly, when the gastric restrictive device 100 is clamped or clipped or fitted to the stomach, the two separate elongated rigid members 112, 114 may be aligned lengthwise to serve as a pair of opposing elongated jaws for holding or engaging or gripping the stomach from two opposite sides of the stomach so as to clamp or clip the stomach to restrict or reduce the size of the stomach. According to various embodiments, the two separate elongated rigid members 112, 114 may be two separate pieces of solid structures that are not directly touching or abutting or in contact with each other when the gastric restrictive device 100 is in a non-closed or opened configuration, for example as shown in FIG. 1A. According to various embodiments, the two separate elongated rigid members 112, 114 may include a first elongated rigid member 112 and a second elongated rigid member 114.

According to various embodiments, the first elongated rigid member 112 of the two separate elongated rigid members 112, 114 may have an elongated engagement side 112a, and the second elongated rigid member 114 of the two separate elongated rigid member 112, 114 may have a corresponding elongated engagement side 114a. Accordingly, when the gastric restrictive device 100 is clamped or clipped or fitted to the stomach, the elongated engagement side 112a of the first elongated rigid member 112 and the corresponding elongated engagement side 114a of the second elongated rigid member 114 may be opposing each other for holding or engaging or gripping the stomach from two opposite sides of the stomach so as to clamp or clip the stomach to restrict or reduce the size of the stomach. According to various embodiments, each of the elongated engagement side 112a of the first elongated rigid member 112 and the corresponding elongated engagement side 114a of the second elongated rigid member 114 may be of an elongated shape including, but not limited to, a rectangular shape, a rounded rectangular shape, a racetrack shape, an oblong shape, an oval shape, an elongated oval shape, or an ellipse shape. According to various embodiments, the elongated engagement side 112a of the first elongated rigid member 112 and the corresponding elongated engagement side 114a of the second elongated rigid member 114 may have a same elongated shape.

According to various embodiments, the gastric restrictive device 100 may include a cushioning arrangement 120. According to various embodiments, the cushioning arrangement 120 may include at least one first elongated inflatable padding 122 lined longitudinally along the first elongated rigid member 112 of the two separate elongated rigid members, and at least one second elongated inflatable padding 124 lined longitudinally along the second elongated rigid member 114 of the two separate elongated rigid members. According to various embodiments, each of the at least one first elongated inflatable padding 122 and the at least one second elongated inflatable padding 124 may be an inflatable stripe or an elongated inflatable balloon. According to various embodiments, the at least one first elongated inflatable padding 122 may be aligned and extending lengthwise along the elongated engagement side 112a of the first elongated rigid member 112. According to various embodiments, the at least one second elongated inflatable padding 124 may be aligned and extending lengthwise along the corresponding elongated engagement side 114a of the second elongated rigid member 114. Accordingly, the at least one first elongated inflatable padding 122 may be running alongside the elongated engagement side 112a of the first elongated rigid member 112 and the at least one second elongated inflatable padding 124 may be running alongside the corresponding elongated engagement side 114a of the second elongated rigid member 114.

According to various embodiments, the cushioning arrangement 120 may include one or two or more first elongated inflatable paddings 122 along the first elongated rigid member 112 of the two separate elongated rigid members. Accordingly, when there are two or more first elongated inflatable paddings 122, the two or more first elongated inflatable paddings 122 may be parallel to each other and lined longitudinally along the first elongated rigid member 112 of the two separate elongated rigid members. According to various embodiments, the cushioning arrangement 120 may include one or two or more second elongated inflatable paddings 124 along the second elongated rigid member 114 of the two separate elongated rigid members. Accordingly, when there are two or more second elongated inflatable paddings 124, the two or more second elongated inflatable paddings 124 may be parallel to each other and lined longitudinally along the second elongated rigid member 114 of the two separate elongated rigid members.

According to various embodiments, when the gastric restrictive device 100 is clamped or clipped or fitted to the stomach, the at least one first elongated inflatable padding 122 of the cushioning arrangement 120 and the at least one second elongated padding 124 of the cushioning arrangement 120 may be parallel and opposing each other. Accordingly, in the closed or folded configuration, the two separate elongated rigid members 112, 114 may be parallel to each other with the at least one first elongated inflatable padding 122 and the at least one second elongated padding 124 directed inwardly so as to be facing each other. Accordingly, the first elongated rigid member 112 and the second elongated rigid member 114 which are in the parallel configuration or disposition may exert a clamping force or pressure on the stomach to restrict the effective size of the stomach without permanently removing any portions of the stomach or permanently changing the anatomy of the stomach. Further, the at least one first elongated inflatable padding 122 and the at least one second elongated padding 124 may form a pair of inner linings of the parallel first and second elongated rigid member 112, 114 that may cushion or soften the contact between the parallel first and second elongated rigid member 112, 114. According to various embodiments, the first elongated rigid member 112 with the at least one first elongated inflatable padding 122 and the second elongated rigid member 114 with the at least one second elongated inflatable padding 124 may be non-movable (or may not be moved) relative to each other when the gastric restrictive device 100 is clamped or clipped or fitted to the stomach. Hence, the gastric restrictive device 100 may be securely or firmly clamped or clipped or fitted to the stomach.

According to various embodiments, the at least one first elongated inflatable padding 122 may be integrally molded or printed or formed with the first elongated rigid member 112 as a single unitary piece. Accordingly, the at least one first elongated inflatable padding 122 may be a thin film or sheet or membrane or layer or ply integrally molded or printed or formed with the first elongated rigid member 112. According to various embodiments, the at least one first elongated inflatable padding 122 and the first elongated rigid member 112 may also be separate pieces integrally joined or coupled or bonded to form a single unit or a single structural whole. Accordingly, the at least one first elongated inflatable padding 122 may be a thin film or sheet or membrane or layer or ply integrally joined or coupled or bonded to the first elongated rigid member 112. According to various embodiments, a perimeter or border or boundary or edges of the at least one first elongated inflatable padding 122 may be joined to or connected to or continuous with the first elongated rigid member 112 such that an empty space between the at least one first elongated inflatable padding 122 and the elongated engagement side 112a of the first elongated rigid member 112 may serve as an inflatable chamber 122a for inflating the at least one first elongated inflatable padding 122. According to various embodiments, the at least one first elongated inflatable padding 122 may be flexible such that the at least one first elongated inflatable padding 122 may be inflated to be maintained in an inflated shape or be deflated to collapse from the inflated shape.

According to various embodiments, the at least one second elongated inflatable padding 124 may be integrally molded or printed or formed with the second elongated rigid member 114 as a single unitary piece. Accordingly, the at least one second elongated inflatable padding 124 may be a thin film or sheet or membrane or layer or ply integrally molded or printed or formed with the second elongated rigid member 114. According to various embodiments, the at least one second elongated inflatable padding 124 and the second elongated rigid member 114 may also be separate pieces integrally joined or coupled or bonded to form a single unit or a single structural whole. Accordingly, the at least one second elongated inflatable padding 124 may be a thin film or sheet or membrane or layer or ply integrally joined or coupled or bonded to the second elongated rigid member 114. According to various embodiments, a perimeter or border or boundary or edges of the at least one second elongated inflatable padding 124 may be joined to or connected to or continuous with the second elongated rigid member 114 such that an empty space between the at least one second elongated inflatable padding 124 and the elongated engagement side 114a of the second elongated rigid member 114 may serve as an inflatable chamber 124a for inflating the at least one second elongated inflatable padding 124. According to various embodiments, the at least one second elongated inflatable padding 124 may be flexible such that the at least one second elongated inflatable padding 124 may be inflated to be maintained in an inflated shape or be deflated to collapse from the inflated shape.

According to various embodiments, the cushioning arrangement 120 may include at least one flexible and extendable hollow tube 126 fluidly interconnecting the at least one first elongated inflatable padding 122 and the at least one second elongated inflatable padding 124. Accordingly, the inflatable chamber 122a of the at least one first elongated inflatable padding 122 and the inflatable chamber 124a of the at least one second elongated inflatable padding 124 may be in fluid communication via the at least one flexible and extendable hollow tube 126. Hence, a fluid may be continuous from the inflatable chamber 122a of the at least one first elongated inflatable padding 122 through a connecting passage 126a of the at least one flexible and extendable hollow tube 126 and to the inflatable chamber 124a of the at least one second elongated inflatable padding 124 so as to connect the inflatable chamber 122a of the at least one first elongated inflatable padding 122, the connecting passage 126a of the at least one flexible and extendable hollow tube 126 and the inflatable chamber 124a of the at least one second elongated inflatable padding 124.

According to various embodiments, the cushioning arrangement 120 may include an inflation point 128 for receiving or feeding or pumping the fluid to inflate the at least one first elongated inflatable padding 122 and/or the at least one second elongated inflatable padding 124. Accordingly, the at least one first elongated inflatable padding 122 and/or the at least one second elongated padding 124 of the cushioning arrangement 120 may be inflated by injecting or feeding or pumping fluid through the inflation point 128. According to various embodiments, the fluid may include, but not limited to, air, water, saline, or water-based solution. According to various embodiments, as represented by dotted lines in FIG. 1A, the inflation point 128 of the cushioning arrangement 120 may be along one or a combination of the at least one first elongated inflatable padding 122, the at least one second elongated inflatable padding 124, and the at least one flexible and extendable hollow tube 126. According to various embodiments, the inflation point 128 may include a valve, such as a one-way check valve or an inflation valve, for inflating the at least one first elongated inflatable padding 122 and/or the at least one second elongated inflatable padding 124.

According to various embodiments, a first end 126b of the at least one flexible and extendable hollow tube 126 may be connected to an end 122b of the at least one first elongated inflatable padding 122 which corresponds to a first longitudinal end 112b of the first elongated rigid member 112, and a second end 126c of the at least one flexible and extendable hollow tube 126 may be connected to an end 124b of the at least one second elongated inflatable padding 124 which correspond to a first longitudinal end 114b of the second elongated rigid member 114. Accordingly, the at least one first elongated inflatable padding 122 may be at the first end 126b of the at least one flexible and extendable hollow tube 126 and the at least one second elongated inflatable padding 124 may be at the second end 126c of the at least one flexible and extendable hollow tube 126, or the at least one flexible and extendable hollow tube 126 may be extending between the end 122b of the at least one first elongated inflatable padding 122 which is co-located with the first longitudinal end 112b of the first elongated rigid member 112 and the end 124b of the at least one second elongated inflatable padding 124 which is co-located with the first longitudinal end 114b of the second elongated rigid member 114.

According to various embodiments, the gastric restrictive device 100 may include an interlocking arrangement 130. According to various embodiments, the interlocking arrangement 130 may include a first interlocking element 132 at a second longitudinal end 112c of the first elongated rigid member 112 and a second interlocking element 134 at a second longitudinal end 114c of the second elongated rigid member 114. According to various embodiments, the first interlocking element 132 and the second interlocking element 134 may respectively be a male interlocking element and a female interlocking element, or vice versa. According to various embodiments, the first interlocking element 132 and the second interlocking element 134 may include, but not limited to, an insert and a hole/groove/slot, a mortise and a tenon, a tongue and a hole/groove/slot, a dovetail and a hole/groove/slot, a key and a hole/groove/slot, a pin and a hole/groove/slot, a tooth and a notch, or a T-shaped key and a T-slot. Accordingly, the interlocking arrangement 130 may be a snap-fit arrangement, or a snap-hook arrangement, or a snap-lock arrangement, or a snap-catch arrangement, or a mesh arrangement, or a pin-slot arrangement, or a key-slot arrangement, or a teeth engagement arrangement.

According to various embodiments, for example, when the first interlocking element 132 is the male interlocking element, the first interlocking element 132 may be extending backward from the second longitudinal end 112c of the first elongated rigid member 112 in a manner so as to overhang above a portion of the at least one first elongated inflatable padding 122 along the first elongated rigid member 112. Accordingly, the first interlocking element 132 may be suspended by a support structure 131 at the second longitudinal end 112c of the first elongated rigid member 112 such that the first interlocking element 132 may hang above the portion of the at least one first elongated inflatable padding 122 and may be extending in a direction parallel to the first elongated rigid member 112 from the second longitudinal end 112c towards the first longitudinal end 112b. According to various embodiments, for example, when the second interlocking element 134 is the female interlocking element, the second interlocking element 134 may extend longitudinally into the second elongated rigid member 114 from the second longitudinal end 114c of the second elongated rigid member 114. Accordingly, the second interlocking element 134 may penetrate or withdraw or go or recede into the second longitudinal end 114c of the second elongated rigid member 114 and extend within the second elongated rigid member 114 in a manner so as to be parallel to the second elongated rigid member 114. According to various embodiments, the first interlocking element 132 and the second interlocking element 134 may be accordingly reversed such that the first interlocking element 132 is the female interlocking element and the second interlocking element 134 is the male interlocking element.

According to various embodiments, the at least one flexible and extendable hollow tube 126 of the cushioning arrangement 120 may be bendable to fold the two separate elongated rigid members 112, 114 towards each other from the non-closed or opened configuration into the closed or folded configuration. Accordingly, due to the flexibility or pliability of the at least one flexible and extendable hollow tube 126 of the cushioning arrangement 120, the at least one flexible and extendable hollow tube 126 may be bent or flexed or folded into a curved or angular form under an application of a bending force. Hence, with the at least one flexible and extendable hollow tube 126 of the cushioning arrangement 120 being bent or flexed or folded, the at least one first elongated inflatable padding 122 of the cushioning arrangement 120 and the at least one second elongated inflatable padding 124 of the cushioning arrangement 120 may be folded toward each other, thereby bringing the two separate elongated rigid members 112, 114 to close towards each other in a folding or pivoting manner with respect to the cushioning arrangement 120.

According to various embodiments, in the non-closed or opened configuration, the first interlocking element 132 of the interlocking arrangement 130 and the second interlocking element 134 of the interlocking arrangement 130 may be spaced apart from each other in a non-engageable disposition. Accordingly, in the non-closed or opened configuration, the two separate elongated rigid members 112, 114, along with the at least one first elongated inflatable padding 122 of the cushioning arrangement 120 and the at least one second elongated inflatable padding 124 of the cushioning arrangement 120, may be in a V-shaped or a straightened (for example as shown in FIG. 1A) configuration or disposition such that the second longitudinal end 112c of the first elongated rigid member 112 and the second longitudinal end 114c of the second elongated rigid member 114 may be spaced apart with a gap or some distance therebetween in order for the first interlocking element 132 and the second interlocking element 134 to be positioned apart so as to be not touching or abutting or in contact or engageable. Hence, in the non-closed or opened configuration, the two separate elongated rigid members 112, 114 may be in a configuration or disposition such that the first interlocking element 132 at the second longitudinal end 112c of the first elongated rigid member 112 and the second interlocking element 134 at the second longitudinal end 114c of the second elongated rigid member 114 may not meet.

According to various embodiments, in the closed or folded configuration (for example as shown in FIG. 1B), the at least one first elongated inflatable padding 122 of the cushioning arrangement 120 and the at least one second elongated padding 124 of the cushioning arrangement 120 may be parallel and opposing each other and the first interlocking element 132 of the interlocking arrangement 130 and the second interlocking element of the interlocking arrangement 130 may be engageable with each other to interlock in the closed or folded configuration. Accordingly, in the closed or folded configuration, the two separate elongated rigid members 112, 114 may be parallel to each other with the at least one first elongated inflatable padding 122 and the at least one second elongated padding 124 directed inwardly so as to be facing each other while the two separate elongated rigid members 112, 114 are directed outwardly. Further, the second longitudinal end 112c of the first elongated rigid member 112 and the second longitudinal end 114c of the second elongated rigid member 114 may be aligned such that the first interlocking element 132 at the second longitudinal end 112c of the first elongated rigid member 112 and the second interlocking element 134 at the second longitudinal end 114c of the second elongated rigid member 114 may be engageable or touching or abutting or in contact. According to various embodiments, when the first interlocking element 132 at the second longitudinal end 112c of the first elongated rigid member 112 and the second interlocking element 134 at the second longitudinal end 114c of the second elongated rigid member 114 are engaged to each other, the gastric restrictive device 100 may be locked in the closed or folded configuration.

According to various embodiments, the gastric restrictive device 100, in the closed or folded configuration, may be locked and clamped/clipped/fitted to the stomach. Accordingly, the first elongated rigid member 112 and the second elongated rigid member 114 which are locked in the parallel configuration or disposition may exert a clamping force or pressure on the stomach to restrict the effective size of the stomach without permanently removing any portions of the stomach or permanently changing the anatomy of the stomach. Further, the at least one first elongated inflatable padding 122 and the at least one second elongated padding 124, which are respectively along the elongated engagement side 112a of the first elongated rigid member 112 and the corresponding elongated engagement side 114a of the second elongated rigid member 114, may form the pair of inner linings of the parallel first and second elongated rigid member 112, 114 that may cushion or soften the contact between the parallel first and second elongated rigid member 112, 114 and the stomach so as to minimize or avoid damage or bruising to the stomach tissue. Hence, the first elongated rigid member 112 with the at least one first elongated inflatable padding 122 and the second elongated rigid member 114 with the at least one second elongated inflatable padding 124 may be cooperatively operable to clamp or clip the stomach in the closed configuration so as to restrict the stomach in a reversible anatomically manner.

According to various embodiments, the at least one flexible and extendable hollow tube 126 of the cushioning arrangement 120 may be extendable along an axis of the at least one flexible and extendable hollow tube 126. FIG. 1C shows a schematic diagram of the gastric restrictive device 100 of FIG. 1B in the closed configuration with the at least one flexible and extendable hollow tube 126 being extended according to various embodiments. Accordingly, when the gastric restrictive device 100 is locked and clamped/clipped/fitted to the stomach in the closed or folded configuration, the at least one flexible and extendable hollow tube 126, which is bent, may be extendable along the curved axis in a manner so as to stretch or lengthen or elongate or extend or expand according to the peristalsis movement of the stomach. Hence, with the at least one flexible and extendable hollow tube 126 of the cushioning arrangement 120 being extendable along its axis, the gastric restrictive device 100 may provide some degree of freedom or flexibility or leeway to accommodate the peristalsis movement of the stomach so as to be compatible with the physiology of the stomach and minimize or eliminate the gastric restrictive device 100 acting antagonistic against the physiology of the stomach for minimizing or avoiding damage or bruising of the stomach tissue.

According to various embodiments, a ratio of a length of the two separate elongated rigid members 112, 114 to a length of the at least one flexible and extendable hollow tube 126 of the cushioning arrangement 120 may be based on the anatomy of the stomach taking into consideration the different muscle thickness and their respective purpose and movement in digestion. For example, the length of the two separate elongated rigid members 112, 114 may be equal to or greater than the length of the at least one flexible and extendable hollow tube 126 of the cushioning arrangement 120, or the length of the two separate elongated rigid members 112, 114 may be two or three or four times or more than the length of the at least one flexible and extendable hollow tube 126 of the cushioning arrangement 120. According to various embodiments, the gastric restrictive device 100 may be locked and clamped/clipped/fitted to the stomach in the closed or folded configuration with the parallel first and second elongated rigid members 112, 114 extending downwards from the fundus of the stomach to the body of the stomach, and the at least one flexible and extendable hollow tube 126 of the cushioning arrangement 120 extending from the parallel first and second elongated rigid members 112, 114 downwards to loop or wrap around a bottom of the body of the stomach.

According to various embodiments, the at least one flexible and extendable hollow tube 126 of the cushioning arrangement 120 may include, but not limited to, a tube with corrugated bellows, or an elastic tube that is flexible and elastic, or an elastic tube with mesh sleeve or braided sleeve.

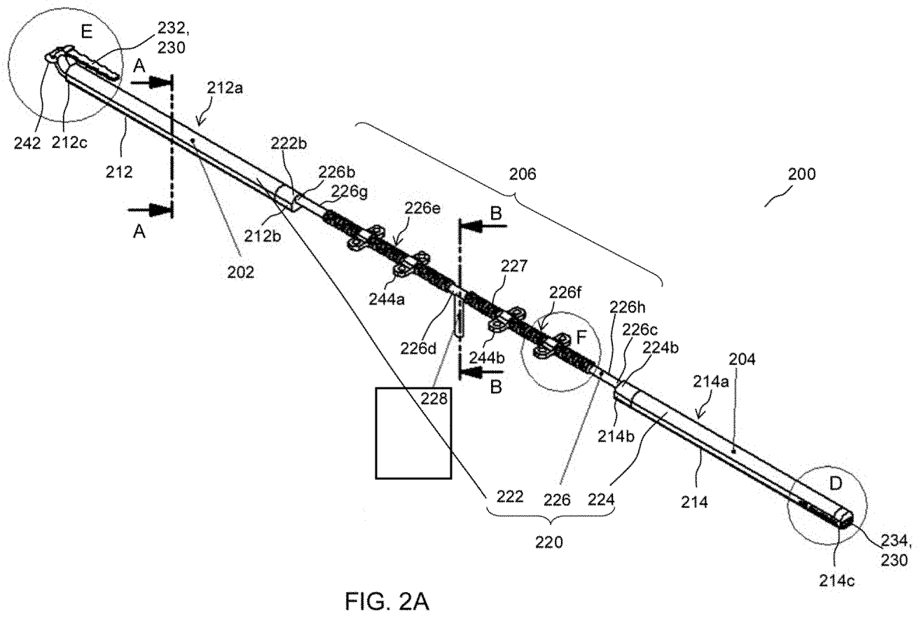

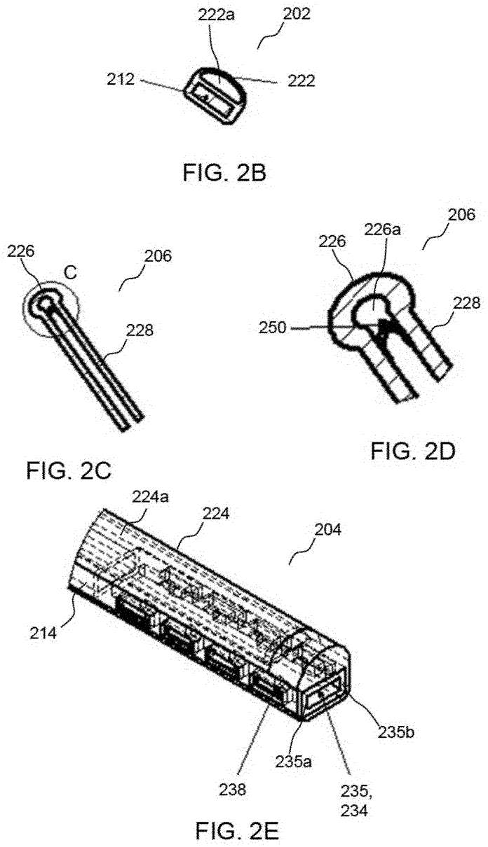

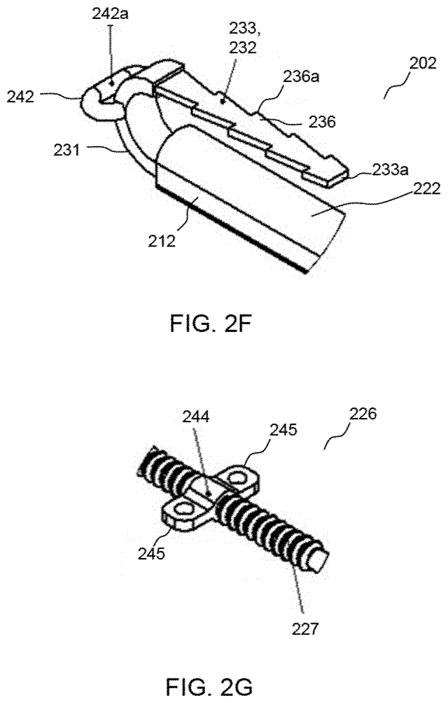

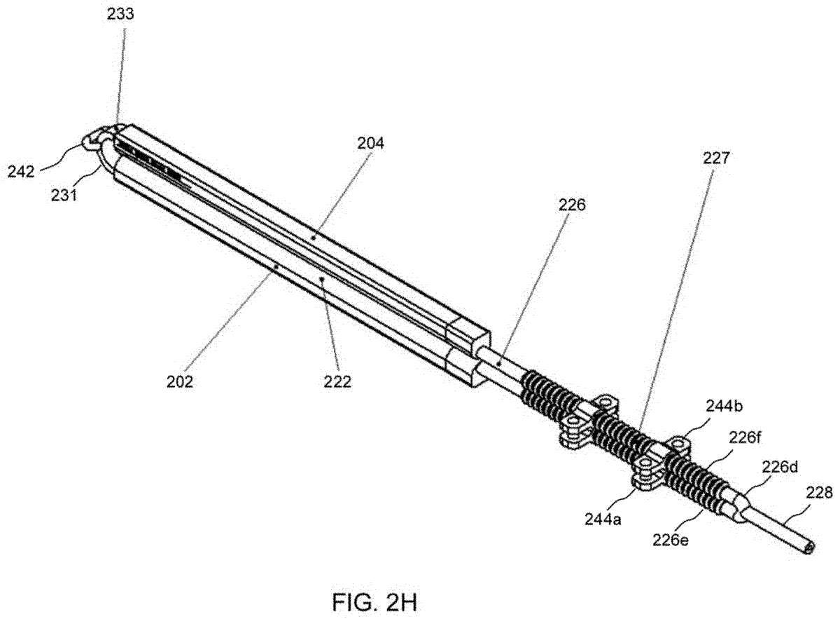

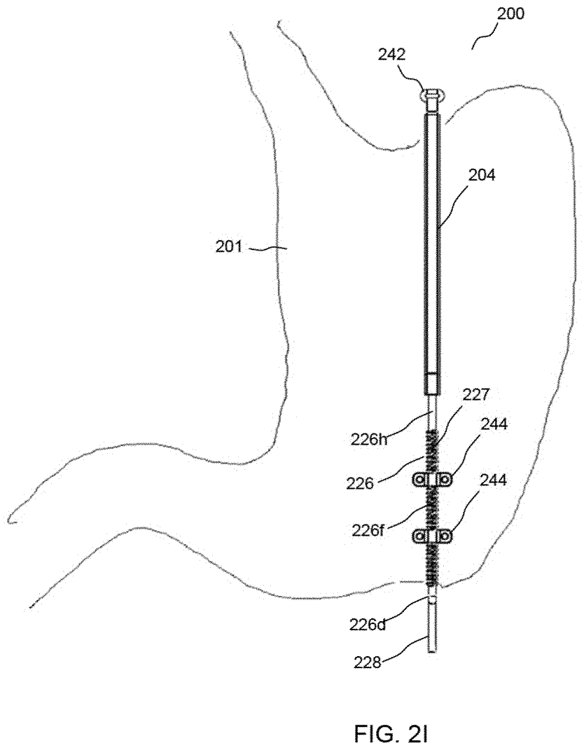

FIG. 2A shows a gastric restrictive device 200, as an example of the gastric restrictive device 100 of FIG. 1A to FIG. 1C, according to various embodiments. FIG. 2B shows an A-A cross-section of the gastric restrictive device 200 of FIG. 2A according to various embodiments. FIG. 2C shows a B-B cross-section of the gastric restrictive device 200 of FIG. 2A according to various embodiments. FIG. 2D shows an enlarged view of a circled portion C of FIG. 2C according to various embodiments. FIG. 2E shows an enlarged view of a circled portion D of FIG. 2A according to various embodiments. FIG. 2F shows an enlarged view of a circled portion E of FIG. 2A according to various embodiments. FIG. 2G shows an enlarged view of a circled portion F of FIG. 2A according to various embodiments. FIG. 2H shows the gastric restrictive device 200 in a closed configuration according to various embodiments. FIG. 2I shows the gastric restrictive device 200 clamped or clipped or fitted to a stomach 201 according to various embodiments. According to various embodiments, the gastric restrictive device 200 is provided to illustrate an example of the gastric restrictive device 100 of FIG. 1A to FIG. 1C. Accordingly, the gastric restrictive device 200 includes all the features and limitations of the gastric restrictive device 100 of FIG. 1A to FIGS. 1C and 1s described in the following with similar reference characters referring to the same/common parts throughout. Further details of the gastric restrictive device 200 as shown are also described in the following.

According to various embodiments, the gastric restrictive device 200 of FIG. 2A may, similar to the gastric restrictive device 100 of FIG. 1A to FIG. 1C, be folded into the closed or folded configuration of FIG. 2H for clamping or clipping or banding of the stomach. According to various embodiments, the gastric restrictive device 200 may be a gastric clamp or a gastric clip or a gastric band or a gastroplasty device. As shown in FIG. 2I, according to various embodiments, the gastric restrictive device 200 in the closed or folded configuration may be configured to restrict the effective size of the stomach 201 without permanently removing any portions of the stomach or permanently changing the anatomy of the stomach.

As shown in FIG. 2A, the gastric restrictive device 200 may include two connected arm parts 202 and 204, each including an elongated rigid member 212, 214 (or solid armour part or body) and at least one elongated inflatable padding 222, 224 (or at least an inflatable part). FIG. 2B shows the A-A cross section of the first arm part 202 including the first elongated rigid member 212 and the at least one first elongated inflatable padding 222. In a similar fashion, the second arm part 204 may include the second elongated rigid member 214 and the at least one second elongated inflatable padding 224. According to various embodiments, these parts collectively or cooperatively perform the function of the gastric restrictive device 200 for clipping the stomach 201 (for example, see FIG. 2I) through inflation and contact.

According to various embodiments, a width or a cross section of the gastric restrictive device 200 along its length may be minimized so that it may pass through a trocar, in a manner so as to be insertable through the trocar in a laparoscopic obesity surgery inside the body, in an unfolded or non-closed or opened configuration.

According to various embodiments, the gastric restrictive device 200 may include a first interlocking element 232 in the form of an elongated insert member 233, which is an extension of a leg part of a U-shape support structure 231 of the first arm 202, wherein the elongated insert member 233 may possess a series of ratchet-like teeth 236a (or wedges). According to various embodiments, the gastric restrictive device 200 may include a second interlocking element 234 in the form of a corresponding elongated hole 235 (or a hollow part or a cavity) at a free end of the second arm 204, wherein the corresponding elongated hole 235 (or the hollow part or the cavity) may possess a series of sockets 238 (or snap hooks or cavities). According to various embodiments, after the gastric restrictive device 200 is inserted into the body, the gastric restrictive device 200 may be folded and the two anus 202, 204 may be placed at the two sides of the stomach tissue and may be fixed together by inserting the first interlocking element 232 into the second interlocking element 234 which together may form an interlocking arrangement 230 (for example, a snap-hook mechanism). Accordingly, the gastric restrictive device 200 may be folded and locked when inside the abdominal cavity so as to be in contact with the outer tissue of the stomach 201 for clamping the fundus and/or body section of the stomach 201.

Referring to FIG. 2A and FIG. 2H, the gastric restrictive device 200 may include the first arm part 202 (or a lower arm) and second arm part 204 (or an upper arm). Each of the first arm part 202 and the second arm part 204 may include the elongated rigid member 212, 214 (or the body) and the at least one elongated inflatable padding 222, 224 (or the inflatable part), as shown in the A-A cross section view of FIG. 2B. According to various embodiments, each of the elongated inflatable paddings 222, 224 (or the inflatable part) may be respectively secured to and supported by the elongated rigid members 212, 214. According to various embodiments, each of the elongated rigid members 212, 214 (or the body) may be fabricated from a firm material such as a biomedical metal. According to various embodiments, each of the elongated inflatable paddings 222, 224 (or the inflatable part) may be fabricated from a flexible material such as medical grade silicone polymer.

Referring to FIG. 2A, according to various embodiments, the elongated inflatable paddings 222, 224 (or the inflatable part) of the arm parts 202, 204 may be connected to a connection portion 206. According to various embodiments, the connection portion 206 may include at least one flexible and extendable hollow tube 226 (or a connecting tube). According to various embodiments, the at least one flexible and extendable hollow tube 226 may include corrugated bellows 227 (or intermittent accordion-like parts) along its tube body. According to various embodiments, the connection portion 206 may include a branch tube 228 (or a side tube) in connection with and extending from a smooth segment 226d of the at least one flexible and extendable hollow tube 226 (or the connecting tube). According to various embodiments, the branch tube 228 may be sufficiently long to extend to the outside of the patient's body through the trocar after the gastric restrictive device 200 is inserted. According to various embodiments, the connection portion 206 may include at least one stomach body securing component 244 (or suture points) positioned along the at least one flexible and extendable hollow tube 226. According to various embodiments, the at least one stomach body securing component 244 (or the suture points) may be positioned and configured to provide secure points for suturing the gastric restrictive device 200 to the patient's stomach. According to various embodiments, the at least one stomach body securing component 244 may be made from a durable material. According to various embodiments, the at least one flexible and extendable hollow tube 226 (or the connecting tube) and the branch tube 228 (or the side tube) may be made from a flexible material such as medical grade silicone polymer to facilitate transitions between a closed (or folded) and the non-closed (or opened or unfolded) configurations of the gastric restrictive device 200.

Referring to FIG. 2E, according to various embodiments, the second arm part 204 may further include an opening on a free end of the second elongated rigid member 214 (or the body), whereby the opening opens to the elongated hole 235 (or the hollow part or the cavity) serving as the second interlocking element 234. According to various embodiments, the elongated hole 235 (or the hollow part or the cavity) may include the series of sockets 236 (or snap hooks or cavities) within. Referring to FIG. 2F, according to various embodiments, a free end of the first elongated rigid member 212 of the first arm part 202 may include the U-shaped support structure 231 supporting the elongated insert member 233 with the series of ratchet-like teeth 236a (or wedges). The elongated insert member 233 may serves as the first interlocking element 232. According to various embodiments, after the gastric restrictive device 200 is inserted into the body, the first arm part 202 and the second arm part 204 may be folded towards each other via the connection portion 206 into the closed (or folded) configuration for clamping two sides of the stomach 201. Accordingly, the first arm part 202 and the second arm part 204 may then be secured by inserting the elongated insert member 233 serving as the first interlocking element 232 into the corresponding elongated hole 235 serving as the second interlocking element 234 to interlock the first aim part 202 and the second arm part 204.

According to various embodiments, the gastric restrictive device 200 may include a securing arrangement 240. According to various embodiments, the securing arrangement 240 may include a stomach fundus securing component 242 (or a fundus suture point) and the at least one stomach body securing component 244 (or suture point). According to various embodiments, once the gastric restrictive device 200 is inserted and folded, it may be fixed by sutures to the stomach at specific locations, for example via the stomach fundus securing component 242 (or the fundus suture point) to the stomach fundus and via several stomach body securing components 244 (or suture points) to the stomach body. Accordingly, the gastric restrictive device 200 may be fixed in position by the use of sutures at selected suturing points along its length. Further, according to various embodiments, the gastric restrictive device 200 may have some flexibility. For example, the corrugated bellows 227 (or the flexible accordion sections) of the at least one flexible and extendable hollow tube 226 (or the connecting tube) may have the capability to elongate or lengthen or extend when the stomach is being filled as well as when it is going through peristalsis movements. Thus, the gastric restrictive device 200 may not overly-constrain at the suture points to the stomach tissue and may not significantly interfere with the peristalsis movement of the stomach and hence the digestion process. According to various embodiments, the stomach fundus securing component 242 may be extending from the U-shaped support structure 231 of the first arm part 202. According to various embodiments, the stomach fundus securing component 242 may be provided proximate to the elongated hole 235 (or hollow part or the cavity) of the second arm part 204 or any other positions along the first arm part 202 and the second arm part 204 as long as it is suitable for the same purpose.

According to various embodiments, the at least one first elongated inflatable padding 222 (or first inflatable part) and the at least one second elongated inflatable padding 224 (or second inflatable part) may be connected by the at least one flexible and extendable hollow tube 226 (or the connecting tube) having the corrugated bellows 227 (or the intermittent accordion-like part). According to various embodiments, a specific amount of solution may be injected to the elongated inflatable paddings 222, 224 (or the inflatable parts) of the two arm parts 202, 204 through the at least one flexible and extendable hollow tube 226 (or the connecting tube) and the branch tube 228 (or the side tube). The branch tube 228 (or the side tube) is only partially shown in FIG. 2A. According to various embodiments, the branch tube 228 (or the side tube) may be sufficiently long to extend to the outside of the patient's body through trocar after inserting the gastric restrictive device 200. According to various embodiments, an embedded one-way check valve 250 may be at a junction point of the at least one flexible and extendable hollow tube 226 and the branch tube 228 to prevent backflow so as to ensure that the injected biocompatible solution is encapsulated inside the gastric restrictive device 200 and may not come in contact with the patient's abdomen. According to various embodiments, the extra length of the branch tube 228 may be cauterized, cut and removed at the end of surgery.

According to various embodiments, a pre-determined amount of biocompatible solution such as saline water may be injected into the elongated inflatable paddings 222, 224 (or the inflatable parts) of the two arm parts 202, 204 though the at least one flexible and extendable hollow tube 226 (or the connecting tube) and/or the branch tube 228 (or the side tube). According to various embodiments, the gastric restrictive device 200 may be pressurized and inflated by injecting the pre-determined amount of biocompatible solution for providing localized sealing at the clamped part of the stomach and hence restrict it to avoid over-eating in a reversible way (without a need to cut a portion of stomach). According to various embodiments, the contact pressure applied on the outer stomach tissue may be adjusted by the amount of biocompatible solution injected into the gastric restrictive device 200, to ensure the sealing function of the gastric restrictive device 200 in consideration with the maximum internal pressure of the human stomach during operation of the gastric restrictive device 200. According to various embodiments, the biocompatible solution may not completely fill the elongated inflatable paddings 222, 224 (or the inflatable parts) and/or the at least one flexible and extendable hollow tube 226 (or the connecting tube). According to various embodiments, the injection of the biocompatible solution may be performed before and/or after the clamping and suturing of the gastric restrictive device 200. According to various embodiments, the embedded one-way check valve 250 may be provided at the connection between the branch tube 228 (or the side tube) and the at least one flexible and extendable hollow tube 226 (or the connecting tube) for preventing the backflow of injected biocompatible solution. Hence, the injected biocompatible solution may be encapsulated inside the elongated inflatable paddings 222, 224 (or the inflatable parts) and/or the at least one flexible and extendable hollow tube 226 (or the connecting tube) of the gastric restrictive device 200 and may not come in contact with the patient's abdomen. According to various embodiments, any unnecessary length of the branch tube 228 (or the side tube) may be cauterized, cut and removed at the end of surgery. According to various embodiments, the cut end of the branch tube 228 may be configured to be coupled to a fluid source for injecting the fluid via the branch tube 228 through the one-way check valve 250 into the at least one flexible and extendable hollow tube 226, and from the at least one flexible and extendable hollow tube 226 into the elongated inflatable paddings 222, 224 for inflating the elongated inflatable paddings 222, 224.

According to various embodiments, when the gastric restrictive device 200 is in the closed or folded configuration (for example see FIG. 2H), the two arm parts 202, 204 may exert a clamping force or pressure on the stomach 201. The elongated inflatable paddings 222, 224 (or the inflatable parts) of the two arm parts 202, 204, may act as cushions between the elongated rigid members 212, 214 (or the device body) and the stomach surface for reducing the overall clamping force or pressure.

According to various embodiments, the corrugated bellows 227 (or the accordion-like parts) of the at least one flexible and extendable hollow tube 226 (or the connecting tube) may have the capability to elongate/lengthen and compress/shorten when the stomach expands and contracts during an eating/digestion cycle as well as when it is going through peristalsis movements. According to various embodiments, when the volume inside the corrugated bellows 227 (or the accordion-like parts) is expanded, additional volume of space in the at least one flexible and extendable hollow tube 226 (or the connecting tube) may become available. The biocompatible solution and air in the elongated inflatable paddings 222, 224 (or the inflatable parts) may flow into the additional vohune of space, and as such, the elongated inflatable paddings 222, 224 (or the inflatable parts) may become partially deflated and exerts a lower clamping pressure on the stomach. In a reversed manner, when the volume inside the corrugated bellows 227 (or the accordion-like parts) is compressed, the biocompatible solution and air may be pushed back into elongated inflatable paddings 222, 224 (or the inflatable parts), and the original clamping pressure may be reinstated. Accordingly, the contact pressure applied on the external tissue of the stomach 201 may thus passively vary during the digestion cycle to accommodate the stomach tissue blood circulation at the contact areas to avoid wound pressure (stomach tissue erosion). Also, as the stomach starts its peristalsis movement to digest the food, the contact pressure may vary with the movement and at certain stage may reduce to accommodate the stomach tissue blood circulation at the contact areas.

For example, when the stomach is expanded during peristalsis movements, the corrugated bellows 227 (or the accordion-like parts) of the at least one flexible and extendable hollow tube 226 (or the connecting tube) may be elongated/lengthened such that the volume inside may be expanded. Accordingly, the fluid in the elongated inflatable paddings 222, 224 (or the inflatable parts) may flow into the expanded volume of the corrugated bellows 227 (or the accordion-like parts) of the at least one flexible and extendable hollow tube 226 (or the connecting tube) causing the elongated inflatable paddings 222, 224 (or the inflatable parts) to be partially deflated which reduces the clamping pressure on the stomach. When the stomach is contracted during peristalsis movements, the corrugated bellows 227 (or the accordion-like parts) of the at least one flexible and extendable hollow tube 226 (or the connecting tube) may be compressed/shortened such that the volume inside may contract. Accordingly, the fluid may be flowed back to the elongated inflatable paddings 222, 224 (or the inflatable parts) so as to expand or dilate the elongated inflatable paddings 222, 224 (or the inflatable parts) which restores the clamping pressure on the stomach.

According to various embodiments, there is provided the gastric restrictive device 200, for example a gastric clamp or a gastric clip or a gastric band or a gastroplasty device. The gastric restrictive device 200 may include the first arm part 202 and the second arm part 204. The gastric restrictive device 200 may include the at least one first elongated inflatable padding 222 (or the first inflatable part) and the at least one second elongated inflatable padding 224 (or the second inflatable part) respectively secured or coupled to the first aim part 202 and the second arm part 204. The gastric restrictive device 200 may include the connection portion 206 including the at least one flexible and extendable hollow tube 226 (or the connecting tube) having the corrugated bellows (or the accordion-like parts), the at least one flexible and extendable hollow tube 226 (or the connecting tube) connected to and in fluid communication with the at least one first elongated inflatable padding 222 (or the first inflatable part) and the at least one second elongated inflatable padding 224 (or the second inflatable part). The gastric restrictive device 200 may include the interlocking arrangement 230 (or the securing mechanism) for interlocking or securing an end of each of the first arm part 202 and the second arm part 204 together. The gastric restrictive device 200 may include the at least one stomach body securing component 244 (o the plurality of suture points) positioned along the connection portion 206 for suturing the gastric restrictive device 200 to the stomach 201.

According to various embodiments, the connection portion 206 may include the branch tube 228 (or the side tube) in fluid communication with the at least one flexible and extendable hollow tube 226 (or the connecting tube) for injection of the biocompatible solution into the connection portion 206, the at least one first elongated inflatable padding 222 (or the first inflatable part) and the at least one second elongated inflatable padding 224 (or the second inflatable part).

According to various embodiments, the one-way check valve 250 may be provided at the connection or junction between the branch tube 228 (or the side tube) and the at least one flexible and extendable hollow tube 226 (or the connecting tube) for preventing backflow of the injected biocompatible solution.

According to various embodiments, the interlocking arrangement 230 (or the securing mechanism) may include the snap-lock arrangement including the series of ratchet-like teeth 236a (or wedges) provided on the elongated insert member 233 extending from the U-shaped support structure 231 of the first arm part 202, and a series of sockets 238 (or the snap hooks or cavities) housed within the corresponding elongated hole (or cavity) provided in the second arm part 204 for receiving and securing engaging or hooking the series of ratchet-like teeth 236a (or wedges).

According to various embodiments, the U-shaped support structure 231 may include the stomach fundus securing component 242 (or the fundus suture point) for specifically suturing the gastric restrictive device 200 to the fundus section of the stomach 201.

According to various embodiments, the gastric restrictive device 200 may, similar to the gastric restrictive device 100 of FIG. 1A to FIG. 1C, include the two separate elongated rigid members 212, 214. Each of the two separate elongated rigid members 212, 214 may respectively be a solid structure which may be stiff and inflexible. According to various embodiments, each of the two separate elongated rigid members 212, 214 may be made of firm material such as a biomedical metal. According to various embodiments, each of the two separate elongated rigid members 212, 214 may be in the form of, including but not limited to, a bar, a rod, a pole, a stripe, a rib, or a stick of solid material. Accordingly, when the gastric restrictive device 200 is clamped or clipped or fitted to the stomach 201, the two separate elongated rigid members 212, 214 may be aligned lengthwise to serve as a pair of opposing elongated jaws for holding or engaging or gripping the stomach 201 from two opposite sides of the stomach 201 so as to clamp or clip the stomach to restrict or reduce the size of the stomach. According to various embodiments, the two separate elongated rigid members 212, 214 may be two separate pieces of solid structures that are not directly touching or abutting or in contact with each other when the gastric restrictive device 100 is in a non-closed or opened configuration, for example as shown in FIG. 2A. According to various embodiments, the two separate elongated rigid members 212, 214 may include the first elongated rigid member 212 and the second elongated rigid member 214.

According to various embodiments, the first elongated rigid member 212 of the two separate elongated rigid members 212, 214 may have an elongated engagement side 212a, and the second elongated rigid member 214 of the two separate elongated rigid member 212, 214 may have a corresponding elongated engagement side 214a. Accordingly, when the gastric restrictive device 200 is clamped or clipped or fitted to the stomach 201, the elongated engagement side 212a of the first elongated rigid member 212 and the corresponding elongated engagement side 214a of the second elongated rigid member 214 may be opposing each other for holding or engaging or gripping the stomach 201 from two opposite sides of the stomach 201 so as to clamp or clip the stomach to restrict or reduce the size of the stomach 201. According to various embodiments, each of the elongated engagement side 212a of the first elongated rigid member 212 and the corresponding elongated engagement side 214a of the second elongated rigid member 214 may be of an elongated shape including, but not limited to, a rectangular shape, a rounded rectangular shape, a racetrack shape, an oblong shape, an oval shape, an elongated oval shape, or an ellipse shape. According to various embodiments, the elongated engagement side 212a of the first elongated rigid member 212 and the corresponding elongated engagement side 214a of the second elongated rigid member 214 may have a same elongated shape.

According to various embodiments, the gastric restrictive device 200 may, similar to the gastric restrictive device 100 of FIG. 1A to FIG. 1C, include a cushioning arrangement 220. According to various embodiments, the cushioning arrangement 220 may include the at least one first elongated inflatable padding 222 lined longitudinally along the first elongated rigid member 212 of the two separate elongated rigid members, and the at least one second elongated inflatable padding 224 lined longitudinally along the second elongated rigid member 214 of the two separate elongated rigid members. According to various embodiments, each of the at least one first elongated inflatable padding 222 and the at least one second elongated inflatable padding 224 may be an inflatable stripe or an elongated inflatable balloon. According to various embodiments, the at least one first elongated inflatable padding 222 may be aligned and extending lengthwise along the elongated engagement side 212a of the first elongated rigid member 212. According to various embodiments, the at least one second elongated inflatable padding 224 may be aligned and extending lengthwise along the corresponding elongated engagement side 214a of the second elongated rigid member 214. Accordingly, the at least one first elongated inflatable padding 222 may be running alongside the elongated engagement side 212a of the first elongated rigid member 212 and the at least one second elongated inflatable padding 224 may be running alongside the corresponding elongated engagement side 214a of the second elongated rigid member 214.

According to various embodiments, the cushioning arrangement 220 may include one or two or more (or at least one) first elongated inflatable paddings 222 along the first elongated rigid member 212 of the two separate elongated rigid members. Accordingly, when there are two or more first elongated inflatable paddings 222 (not shown), the two or more first elongated inflatable paddings 222 may be parallel to each other and lined longitudinally along the first elongated rigid member 212 of the two separate elongated rigid members. According to various embodiments, the cushioning arrangement 220 may include one or two or more (at least one) second elongated inflatable paddings 224 along the second elongated rigid member 214 of the two separate elongated rigid members. Accordingly, when there are two or more second elongated inflatable paddings 224 (not shown), the two or more second elongated inflatable paddings 224 may be parallel to each other and lined longitudinally along the second elongated rigid member 214 of the two separate elongated rigid members.

According to various embodiments, when the gastric restrictive device 200 is clamped or clipped or fitted to the stomach 201, the at least one first elongated inflatable padding 222 of the cushioning arrangement 220 and the at least one second elongated padding 224 of the cushioning arrangement 220 may be parallel and opposing each other. Accordingly, in the closed or folded configuration, the two separate elongated rigid members 212, 214 may be parallel to each other with the at least one first elongated inflatable padding 222 and the at least one second elongated padding 224 directed inwardly so as to be facing each other. Accordingly, the first elongated rigid member 212 and the second elongated rigid member 214 which are in the parallel configuration or disposition may exert a clamping force or pressure on the stomach 201 to restrict the effective size of the stomach 201 without permanently removing any portions of the stomach or permanently changing the anatomy of the stomach 201. Further, the at least one first elongated inflatable padding 222 and the at least one second elongated padding 224 may form a pair of inner linings of the parallel first and second elongated rigid member 212, 214 that may cushion or soften the contact between the parallel first and second elongated rigid member 212, 214. According to various embodiments, the first elongated rigid member 212 with the at least one first elongated inflatable padding 222 and the second elongated rigid member 214 with the at least one second elongated inflatable padding 224 may be non-movable (or may not be moved) relative to each other when the gastric restrictive device 200 is clamped or clipped or fitted to the stomach 201. Hence, the gastric restrictive device 200 may be securely or firmly clamped or clipped or fitted to the stomach 201.

According to various embodiments, each of the at least one first elongated inflatable padding 222 and the at least one second elongated inflatable padding 224 may be integrally molded or printed or formed with the first elongated rigid member 212 and the second elongated rigid member 214 respectively as a single unitary piece. Accordingly, each of the at least one first elongated inflatable padding 222 and the at least one second elongated inflatable padding 224 may be a thin film or sheet or membrane or layer or ply integrally molded or printed or formed with the first elongated rigid member 212 and the second elongated rigid member 214 respectively. According to various embodiments, each pair of the elongated inflatable paddings 222, 224 and the elongated rigid members 212, 214 may also be separate pieces integrally joined or coupled or bonded to form a single unit or a single structural whole. Accordingly, each of the at least one first elongated inflatable padding 222 and the at least one second elongated inflatable padding 224 may be a thin film or sheet or membrane or layer or ply integrally joined or coupled or bonded to the first elongated rigid member 212 and the second elongated rigid member 214 respectively.

According to various embodiments, a perimeter or border or boundary or edges of the at least one first elongated inflatable padding 222 and/or the at least one second elongated inflatable padding 224 may be joined to or connected to or continuous with the first elongated rigid member 212 and/or the second elongated rigid member 214 respectively such that an empty space between each pair of the elongated inflatable paddings 222, 224 and the elongated engagement sides 212a, 214a of the elongated rigid members 212, 214 may serve as an inflatable chamber 222a, 224a for inflating the elongated inflatable paddings 222, 224 respectively. According to various embodiments, each of the at least one first elongated inflatable padding 222 and the at least one second elongated inflatable padding 224 may be flexible such that each of the at least one first elongated inflatable padding 222 and the at least one second elongated inflatable padding 224 may be inflated to be maintained in an inflated shape or be deflated to collapse from the inflated shape. Each of the at least one first elongated inflatable padding 222 and the at least one second elongated inflatable padding 224 may be made of medical grade silicone polymer.

According to various embodiments, each of the at least one first elongated inflatable padding 222 and the at least one second elongated inflatable padding 224 may be molded or printed or formed as a single unitary elongated structure having two parallel elongated hollow spaces therein. According to various embodiments, the first elongated rigid member 212 and the second elongated rigid member 214 may respectively be fitted in one of the two parallel elongated hollow spaces of each of the at least one first elongated inflatable padding 222 and the at least one second elongated inflatable padding 224 so as to serve as an inner-rigid-core therein. Accordingly, each of the at least one first elongated inflatable padding 222 and the at least one second elongated inflatable padding 224 may respectively wrapped the first elongated rigid member 212 or the second elongated rigid member 214 within one of the two parallel elongated hollow spaces. Hence, each of the at least one first elongated inflatable padding 222 and the at least one second elongated inflatable padding 224 may have dual or twin or double lumen that are parallel to each other, whereby the first elongated rigid member 212 or the second elongated rigid member 214 may respectively be inserted in one of the dual or twin or double lumen such that the at least one first elongated inflatable padding 222 or the at least one second elongated inflatable padding 224 may respectively be aligned and extending lengthwise along the first elongated rigid member 212 or the second elongated rigid member 214. According to various embodiments, the remaining empty one of the two parallel elongated hollow spaces (or the dual or twin or double lumen) of each of the at least one first elongated inflatable padding 222 and the at least one second elongated inflatable padding 224 may respectively serve as the inflatable chamber 222a of the at least one first elongated inflatable padding 222 or the inflatable chamber 224a of the at least one second elongated inflatable padding 224.

According to various embodiments, the cushioning arrangement 220 may include the at least one flexible and extendable hollow tube 226 fluidly interconnecting the at least one first elongated inflatable padding 222 and the at least one second elongated inflatable padding 224. Accordingly, the inflatable chamber 222a of the at least one first elongated inflatable padding 222 and the inflatable chamber 224a of the at least one second elongated inflatable padding 224 may be in fluid communication via the at least one flexible and extendable hollow tube 226. Hence, a fluid may be continuous from the inflatable chamber 222a of the at least one first elongated inflatable padding 222 through a connecting passage 226a of the at least one flexible and extendable hollow tube 226 and to the inflatable chamber 224a of the at least one second elongated inflatable padding 224 so as to connect the inflatable chamber 222a of the at least one first elongated inflatable padding 222, the connecting passage 226a of the at least one flexible and extendable hollow tube 226 and the inflatable chamber 224a of the at least one second elongated inflatable padding 224.

According to various embodiments, the cushioning arrangement 220 may include the branch tube 228 serving as an inflation point for receiving or feeding or pumping the fluid to inflate the at least one first elongated inflatable padding 222 and/or the at least one second elongated inflatable padding 224 and/or the at least one flexible and extendable hollow tube 226. Accordingly, the fluid may be injected via the branch tube 228 for inflating the at least one first elongated inflatable padding 222 and/or the at least one second elongated inflatable padding 224. According to various embodiments, the fluid may include, but not limited to, air, water, saline, or water-based solution. According to various embodiments, as represented by dotted lines in FIG. 2A, the branch tube 228 of the cushioning arrangement 220 may be along the at least one flexible and extendable hollow tube 226. As shown in FIG. 2D, according to various embodiments, the branch tube 228 may include a valve, such as the one-way check valve 250, for inflating the at least one first elongated inflatable padding 222 and/or the at least one second elongated inflatable padding 224 and/or the at least one flexible and extendable hollow tube 226 in a manner so as to prevent backflow. According to various embodiments, an end of the branch tube 228 opposite the one-way check valve 250 may be configured for coupling with the fluid source for injecting the fluid via the branch tube 228 through the one-way check valve 250 into the at least one flexible and extendable hollow tube 226, and from the at least one flexible and extendable hollow tube 226 into the at least one first elongated inflatable padding 222 and/or the at least one second elongated inflatable padding 224.

According to various embodiments, a first end 226b of the at least one flexible and extendable hollow tube 226 may be connected to an end 222b of the at least one first elongated inflatable padding 222 which corresponds to a first longitudinal end 212b of the first elongated rigid member 212, and a second end 226c of the at least one flexible and extendable hollow tube 226 may be connected to an end 224b of the at least one second elongated inflatable padding 224 which correspond to a first longitudinal end 214b of the second elongated rigid member 214. Accordingly, the at least one first elongated inflatable padding 222 may be at the first end 226b of the at least one flexible and extendable hollow tube 226 and the at least one second elongated inflatable padding 224 may be at the second end 226c of the at least one flexible and extendable hollow tube 226, or the at least one flexible and extendable hollow tube 226 may be extending between the end 222b of the at least one first elongated inflatable padding 222 which is co-located with the first longitudinal end 212b of the first elongated rigid member 212 and the end 224b of the at least one second elongated inflatable padding 224 which is co-located with the first longitudinal end 214b of the second elongated rigid member 214.

According to various embodiments, the at least one flexible and extendable hollow tube 226 of the cushioning arrangement 220 may have the corrugated bellows 227. According to various embodiments, the at least one flexible and extendable hollow tube 226 of the cushioning arrangement 220 may include a first corrugated bellows segment 226e, a second corrugated bellows segment 226f, and the smooth segment 226d extending between the first corrugated bellows segment 226e and the second corrugated bellows segment 226f. Accordingly, the at least one flexible and extendable hollow tube 226 may include the first corrugated bellows segment 226e, the smooth segment 226d, and the second corrugated bellows segment 226f in sequence in the lengthwise direction. Hence, the at least one flexible and extendable hollow tube 226 may include the first corrugated bellows segment 226e followed by the smooth segment 226d, and followed by the second corrugated bellows segment 226f along a length of the at least one flexible and extendable hollow tube 226. According to various embodiments, the smooth segment 226d may be without the corrugated bellows 227. According to various embodiments, the smooth segment may correspond to a middle section of the at least one flexible and extendable hollow tube 226, while the first corrugated bellows segment 226e and the second conugated bellows segment 226f may be respectively on two sides of the middle section of the at least one flexible and extendable hollow tube 226. According to various embodiments, the first corrugated bellows segment 226e and the second corrugated bellows segment 226f may be of a same length. According to various embodiments, a length of the smooth segment 226d may be shorter than the length of the first corrugated bellows segment 226e and/or the second corrugated bellows segment 226f. According to various embodiments, the at least one flexible and extendable hollow tube 226 of the cushioning arrangement 220 may include a first connection segment 226g at the first end 226b of the at least one flexible and extendable hollow tube 226 and a second connection segment 226h at the second end 226c of the at least one flexible and extendable hollow tube 226. According to various embodiments, the first connection segment 226g and the second connection segment 226h may be smooth and without the corrugated bellows 227.

According to various embodiments, the branch tube 228 may be extending from a side of the smooth segment 226d of the at least one flexible and extendable hollow tube 226. According to various embodiments, the branch tube 228 may be fluidly connected or in fluid communication with the smooth segment 226d of the at least one flexible and extendable hollow tube 226. According to various embodiments, the branch tube 228 and the at least one flexible and extendable hollow tube 226 may form a T-shaped or a Y-shaped junction. Accordingly, the branch tube 228 may be extending perpendicularly from the smooth segment 226d of the at least one flexible and extendable hollow tube 226 so as to form the T-shaped junction, or the branch tube 228 may be extending at an angle with respect to a longitudinal axis of the smooth segment 226d of the at least one flexible and extendable hollow tube 226 to form a Y-shaped junction. According to various embodiments, the branch tube 228 may include the one-way check valve 250 for one-way fluid flow from the branch tube 228 into the smooth segment 226d of the at least one flexible and extendable hollow tube 226. Accordingly, fluid may flow in only one direction from the branch tube 228 into the at least one flexible and extendable hollow tube 226 as the one-way check valve 250 may prevent any backflow from the at least one flexible and extendable hollow tube 226 to the branch tube 228. According to various embodiments, the one-way check valve 250 may be located at an end of the branch tube 228 that is in fluid connection with the smooth segment 226d of the at least one flexible and extendable hollow tube 226. Accordingly, the one-way check valve 250 may be at the position where the branch tube 228 opens into the at least one flexible and extendable hollow tube 226.

According to various embodiments, the gastric restrictive device 200 may include the interlocking arrangement 230. According to various embodiments, the interlocking arrangement 230 may include the first interlocking element 232 at a second longitudinal end 212c of the first elongated rigid member 212 and the second interlocking element 234 at a second longitudinal end 214c of the second elongated rigid member 214. According to various embodiments, the first interlocking element 232 and the second interlocking element 234 may respectively be a male interlocking element and a female interlocking element, or vice versa.

According to various embodiments, the first interlocking element 232 of the interlocking arrangement 230 may include the elongated insert member 233. According to various embodiments, the elongated insert member 233 may be extending backward from the second longitudinal end 212c of the first elongated rigid member 212 in a manner so as to overhang above a portion of the at least one first elongated inflatable padding 222 along the first elongated rigid member 212. Accordingly, the first interlocking element 232 in the form of the elongated insert member 233 may be suspended by the support structure 231 at the second longitudinal end 212c of the first elongated rigid member 212 such that the first interlocking element 232 may hang above the portion of the at least one first elongated inflatable padding 222 and may be extending in a direction parallel to the first elongated rigid member 212 from the second longitudinal end 212c towards the first longitudinal end 212b. According to various embodiments, the support structure 231 may be a U-shaped structure, a C-shaped structure, a curved structure, or an arcuate structure. Accordingly, an end (or leg) of the support structure 231 may be connected to the second longitudinal end 212c of the first elongated rigid member 212 and another end (or leg) of the support structure 231 may be connected to the elongated insert member 233 serving as the first interlocking element 232.

According to various embodiments, the second interlocking element 234 of the interlocking arrangement 230 may include the corresponding elongated hole 235. According to various embodiments, the corresponding elongated hole 235 may be extending longitudinally into the second elongated rigid member 214 from the second longitudinal end 214c of the second elongated rigid member 214. Accordingly, the corresponding elongated hole 235 may penetrate or withdraw or go or recede into the second longitudinal end 214c of the second elongated rigid member 214 and extend within the second elongated rigid member 214 in a manner so as to be parallel to the second elongated rigid member 214.

According to various embodiments, the first interlocking element 232 and the second interlocking element 234 may be accordingly reversed.

According to various embodiments, the elongated insert member 233 of the first elongated rigid member 212 may include at least one tooth 236 along the elongated insert member 233. Accordingly, the at least one tooth 236 may include a ratchet-like tooth or a wedge-like tooth. According to various embodiments, the corresponding elongated hole 235 of the second elongated rigid member 214 may include at least a corresponding socket 238 for receiving the at least one tooth 236 of the elongated insert member 233 when the elongated insert member 233 of the first elongated rigid member 212 is inserted into the corresponding elongated hole 235 of the second elongated rigid member 214. According to various embodiments, the at least one tooth 236 and the corresponding socket 238 may provide a more secure or firm interlock between the elongated insert member 233 of the first elongated rigid member 212 and the corresponding elongated hole 235 of the second elongated rigid member 214.

According to various embodiments, the elongated insert member 233 of the first elongated rigid member 212 may include an elongated plate-like structure 233a having two rows of ratchet-like teeth 236a respectively along two opposite longitudinal sides of the elongated plate-like structure 233a. Accordingly, the ratchet-like teeth 236a may be protruding sideways from the two opposite longitudinal sides of the elongated plate-like structure 233a. According to various embodiments, the two rows of ratchet-like teeth 236a may have mirror symmetry about a longitudinal axis of the elongated plate-like structure 233a. According to various embodiments, each row of ratchet-like teeth 236a may be distributed at regular interval along respective longitudinal sides of the elongated plate-like structure 233a.

According to various embodiments, the corresponding elongated hole 235 of the second elongated rigid member 214 may include a rectangular cross-section having two rows of sockets 238 respectively along two opposing longitudinal sidewalls 235a, 235b of the corresponding elongated hole 235. Accordingly, the corresponding elongated hole 235 may include two opposing rows of sockets 238 along the length of the corresponding elongated hole 235. According to various embodiments, each socket 238 may be a through-hole or a recess in respective sidewalls 235a, 235b. According to various embodiments, the two rows of sockets 238 may have mirror symmetry about a longitudinal axis of the elongated hole 235. According to various embodiments, each row of sockets 238 may be distributed at regular interval along respective longitudinal sidewalls 235a, 235b of the elongated hole 235.

According to various embodiments, the at least one flexible and extendable hollow tube 226 of the cushioning arrangement 220 may be bendable to fold the two separate elongated rigid members 212, 214 towards each other from the non-closed or opened configuration into the closed or folded configuration. According to various embodiments, the at least one flexible and extendable hollow tube 226 of the cushioning arrangement 220 may be bent or flexed or folded along any segment of the at least one flexible and extendable hollow tube 226 of the cushioning arrangement 220. For example, according to various embodiments, the smooth segment 226d of the at least one flexible and extendable hollow tube 226 may be flexible such that the at least one flexible and extendable hollow tube 226 may bend along the smooth segment 226d of the at least one flexible and extendable hollow tube 226. Accordingly, due to the flexibility or pliability of the at least one flexible and extendable hollow tube 226 of the cushioning arrangement 220, the at least one flexible and extendable hollow tube 226 may be bent or flexed or folded along the smooth segment 226d of the at least one flexible and extendable hollow tube 226 into a curved or angular form under an application of a bending force. Hence, with the at least one flexible and extendable hollow tube 226 of the cushioning arrangement 220 being bent or flexed or folded along the smooth segment 226d of the at least one flexible and extendable hollow tube 226, the at least one first elongated inflatable padding 222 of the cushioning arrangement 220 and the at least one second elongated inflatable padding 224 of the cushioning arrangement 220 may be folded toward each other, thereby bringing the two separate elongated rigid members 212, 214 to close towards each other in a folding or pivoting manner with respect to the bending of the smooth segment 226d of the at least one flexible and extendable hollow tube 226.

According to various embodiments, in the non-closed or opened configuration, the elongated insert member 233 serving as the first interlocking element 232 and the corresponding elongated hole 235 serving as the second interlocking element 234 may be spaced apart from each other in a non-engageable disposition such that the elongated insert member 233 is not insertable or inserted into the corresponding elongated hole 235. Accordingly, in the non-closed or opened configuration, the two separate elongated rigid members 212, 214, along with the at least one first elongated inflatable padding 222 of the cushioning arrangement 220 and the at least one second elongated inflatable padding 224 of the cushioning arrangement 220, may be in a V-shaped or a straightened (for example as shown in FIG. 2A) configuration or disposition such that the second longitudinal end 212c of the first elongated rigid member 212 and the second longitudinal end 214c of the second elongated rigid member 214 may be spaced apart with a gap or some distance therebetween in order for the elongated insert member 233 and the corresponding elongated hole 235 to be positioned apart so as to be not touching or abutting or in contact or engageable or insertable. Hence, in the non-closed or opened configuration, the two separate elongated rigid members 212, 214 may be in a configuration or disposition such that the elongated insert member 233 at the second longitudinal end 212c of the first elongated rigid member 212 and the corresponding elongated hole 235 at the second longitudinal end 214c of the second elongated rigid member 214 may not meet or be insertable or inserted.

According to various embodiments, in the closed or folded configuration (for example as shown in FIG. 2H), the at least one first elongated padding 222 of the cushioning arrangement 220 and the at least one second elongated padding 224 of the cushioning arrangement 220 may be parallel and opposing each other, and the elongated insert member 233 serving as the first interlocking element 232 and the corresponding elongated hole 235 serving as the second interlocking element 234 may be engageable or insertable with each other to interlock in the closed or folded configuration. Accordingly, in the closed or folded configuration, the two separate elongated rigid members 212, 214 may be parallel to each other with the at least one first elongated inflatable padding 222 and the at least one second elongated padding 224 directed inwardly so as to be facing each other while the two separate elongated rigid members 212, 214 are directed outwardly. Further, the second longitudinal end 212c of the first elongated rigid member 212 and the second longitudinal end 214c of the second elongated rigid member 214 may be aligned such that the elongated insert member 233 at the second longitudinal end 212c of the first elongated rigid member 212 and the corresponding elongated hole 235 at the second longitudinal end 214c of the second elongated rigid member 214 may be engageable or insertable or touching or abutting or in contact. According to various embodiments, when the elongated insert member 233 at the second longitudinal end 212c of the first elongated rigid member 212 and the corresponding elongated hole 235 at the second longitudinal end 214c of the second elongated rigid member 214 are engaged or inserted to each other, the gastric restrictive device 200 may be locked in the closed or folded configuration.

According to various embodiments, the gastric restrictive device 200, in the closed or folded configuration, may be locked and clamped/clipped/fitted to the stomach 201. Accordingly, the first elongated rigid member 212 and the second elongated rigid member 214 which are locked in the parallel configuration or disposition may exert a clamping force or pressure on the stomach 201 to restrict the effective size of the stomach 201 without permanently removing any portions of the stomach or permanently changing the anatomy of the stomach. Further, the at least one first elongated inflatable padding 222 and the at least one second elongated padding 224, which are respectively along the elongated engagement side 212a of the first elongated rigid member 212 and the corresponding elongated engagement side 214a of the second elongated rigid member 214, may form a pair of inner linings of the parallel first and second elongated rigid member 212, 214 that may cushion or soften the contact between the parallel first and second elongated rigid member 212, 214 and the stomach so as to minimize or avoid damage or bruising to the stomach tissue. Hence, the first elongated rigid member 212 with the at least one first elongated inflatable padding 222 and the second elongated rigid member 214 with the at least one second elongated inflatable padding 224 may be cooperatively operable to clamp the stomach 201 in the closed configuration so as to restrict the stomach in a reversible anatomically manner.

According to various embodiments, the first corrugated bellows segment 226e of the at least one flexible and extendable hollow tube 226 and the second corrugated bellows segment 226f of the at least one flexible and extendable hollow tube 226 may be extendable or lengthen along respective axis. Accordingly, when the gastric restrictive device 200 is locked and clamped/clipped/fitted to the stomach 201 in the closed or folded configuration, the first corrugated bellows segment 226e of the at least one flexible and extendable hollow tube 226 and the second corrugated bellows segment 226f of the at least one flexible and extendable hollow tube 226 may be extendable or lengthen along the respective axis in a manner so as to stretch or lengthen or elongate or extend or expand the at least one flexible and extendable hollow tube 226 according to the peristalsis movement of the stomach 201. Hence, with the at least one flexible and extendable hollow tube 226 of the cushioning arrangement 220 being extendable, the gastric restrictive device 200 may provide some degree of freedom or flexibility or leeway to accommodate the peristalsis movement of the stomach 201 so as to be compatible with the physiology of the stomach 201 and minimize or eliminate the gastric restrictive device 200 acting antagonistic against the physiology of the stomach 201 for minimising or avoiding damage or bruising of the stomach tissue.

According to various embodiments, a ratio of a length of the two separate elongated rigid members 212, 214 to a length of the first corrugated bellows segment 226e or the second corrugated bellows segment 226 of the at least one flexible and extendable hollow tube 226 may be based on the anatomy of the stomach 201 taking into consideration the different muscle thickness and their respective purpose and movement in digestion. For example, the length of the two separate elongated rigid members 212, 214 may be equal to or greater than the length of the first comigated bellows segment 226e or the second corrugated bellows segment 226 of the at least one flexible and extendable hollow tube 226, or the length of the two separate elongated rigid members 212, 214 may be two or three or four times or more than the length of the first corrugated bellows segment 226e or the second corrugated bellows segment 226 of the at least one flexible and extendable hollow tube 226. According to various embodiments, the gastric restrictive device 200 may be locked and clamped/clipped/fitted to the stomach in the closed or folded configuration with the parallel first and second elongated rigid members 212, 214 extending downwards from the fundus of the stomach 201 to the body of the stomach 201, and the at least one flexible and extendable hollow tube 226 of the cushioning arrangement 220 extending from the parallel first and second elongated rigid members 212, 214 downwards to loop or wrap around a bottom of the body of the stomach 201.

According to various embodiments, the securing arrangement 240 of the gastric restrictive device 200 may include the stomach fundus securing component 242 along the first elongated rigid member 212 or the second elongated rigid member 214 for fixing, e.g. via suturing, to the fundus section of the stomach 201. Accordingly, when the gastric restrictive device 200 is folded and interlocked, the stomach fundus securing component 242 of the gastric restrictive device 200 may be attached to the fundus section of the stomach 201 via the stomach fundus securing component 242. In this manner, the gastric restrictive device 200 may prevent the first elongated rigid member 212 or the second elongated rigid member 214 of the gastric restrictive device 200 from shifting or being displaced after the gastric restrictive device 200 is clamped or clipped or fitted to the stomach 201. As shown in FIG. 2A, according to various embodiments, the stomach fundus securing component 242 may be at the second longitudinal end 212c of the first elongated rigid member 212. Accordingly, when the gastric restrictive device 200 is clamped or clipped or fitted to the stomach 201, the gastric restrictive device 200 may be aligned with the second longitudinal end 212c of the first elongated rigid member 212 directed towards the fundus section of the stomach 201 such that the stomach fundus securing component 242 at the second longitudinal end 212c of the first elongated rigid member 212 may be fixed or sutured to the fundus section of the stomach 201.

According to various embodiments, the stomach fundus securing component 242 may include an eyelet structure 242a. According to various embodiments, the eyelet structure 242a may be protruding from the second longitudinal end 212c of the first elongated rigid member 212 in a direction parallel to the longitudinal axis of the first elongated rigid member 212. According to various embodiments, the eyelet structure 242a may be protruding from the support structure 231 at the second longitudinal end 212c of the first elongated rigid member 212. According to various embodiments, the eyelet structure 242a may be coupled to a middle of the support structure 231 at the second longitudinal end 212c of the first elongated rigid member 212. According to various embodiments, the eyelet structure 242a may be integrally molded or printed or formed with the support structure 231, or may be integrally joined or coupled or bonded to the support structure 231. According to various embodiments, when the gastric restrictive device 200 is clamped or clipped or fitted to the stomach 201, the gastric restrictive device 200 may be fixed to the fundus section of the stomach 201 by suturing through the eyelet structure 242a.

According to various embodiments, the securing arrangement 240 of the gastric restrictive device 200 may include at least one stomach body securing component 244 along the at least one flexible and extendable hollow tube 226 of the cushioning arrangement 220 and interposed between the first end 226b and the second end 226c of the at least one flexible and extendable hollow tube 226. Accordingly, the at least one stomach body securing component 244 may be located at any position along the length of the at least one flexible and extendable hollow tube 226. Hence, when the gastric restrictive device 200 is folded and interlocked, the at least one stomach body securing component 244 of the gastric restrictive device 200 may be attached, for example by suturing, to the body section of the stomach 201 via the at least one stomach body securing component 244. In this manner, the gastric restrictive device 200 may prevent the at least one flexible and extendable hollow tube 226 of the gastric restrictive device 200 from shifting or being displaced after the gastric restrictive device 200 is clamped or clipped or fitted to the stomach 201.

According to various embodiments, the at least one stomach body securing component 244 may include a fixing lug 245 extending sideways from the at least one flexible and extendable hollow tube 226 of the cushioning arrangement 220. Accordingly, the fixing lug 245 may be extending perpendicularly from a side of the at least one flexible and extendable hollow tube 226 of the cushioning arrangement 220. According to various embodiments, the fixing lug 245 may include a flat structure and a hole. According to various embodiments, the hole of the fixing lug 245 may be perpendicular to the at least one flexible and extendable hollow tube 226. Accordingly, the fixing lug 245 may be fixed to the body section of the stomach 201 by suturing through the hole of the fixing lug 245.

According to various embodiments, the at least one stomach body securing component 244 may include a pair of fixing lugs 245 extending sideways in opposite directions from a point along the at least one flexible and extendable hollow tube 226 of the cushioning arrangement 220. Accordingly, the at least one stomach body securing component 244 may include two fixing lugs 245 extending from two opposite sides of the at least one flexible and extendable hollow tube 226. Hence, the two fixing lugs 245 may be on two opposite sides across the at least one flexible and extendable hollow tube 226. Hence, the at least one flexible and extendable hollow tube 226 may be fixed to the body section of the stomach 201 by suturing the two fixing lugs 245 on two opposite sides of the at least one flexible and extendable hollow tube 226.

According to various embodiments, the at least one stomach body securing component 244 may be along the first corrugated bellows segment 226e or the second corrugated bellows segment 226f of the at least one flexible and extendable hollow tube 226 of the cushioning arrangement 220. Accordingly, the at least one stomach body securing component 244 may be disposed at the first corrugated bellows segment 226e or the second corrugated bellows segment 226f. Hence, the at least one stomach body securing component 244 may fixed, for example by suturing, the first corrugated bellows segment 226e or the second corrugated bellows segment 226f of the at least one flexible and extendable hollow tube 226 to the body section of the stomach 201 to prevent the first corrugated bellows segment 226e or the second corrugated bellows segment 226f of the at least one flexible and extendable hollow tube 226 from shifting or being displaced after the gastric restrictive device 200 is clamped or clipped or fitted to the stomach 201.

According to various embodiments, the securing arrangement 240 may include a first stomach body securing component 244a along the first corrugated bellows segment 226e of the at least one flexible and extendable hollow tube 226 and a second stomach body securing component 244b along the second corrugated bellows segment 226f of the at least one flexible and extendable hollow tube 226. Accordingly, the securing arrangement 240 may include at least one first stomach body securing component 244a along the first corrugated bellows segment 226e and at least one second stomach body securing component 244b along the second corrugated bellows segment 226f. According to various embodiments, when the gastric restrictive device 200 is clamped or clipped or fitted to the stomach 201, the securing arrangement 240 may respectively be fixed, for example by suturing, the first corrugated bellows segment 226e and the second corrugated bellows segment 226f to two opposite sides of the stomach 201 via the first stomach body securing component 244a and the second stomach body securing component 244b in a manner so as to prevent the first corrugated bellows segment 226e and the second corrugated bellows segment 226f on both sides of the stomach 201 from shifting or being displaced.

As shown in FIG. 2A, the securing arrangement 240 may include two first stomach body securing component 244a along the first corrugated bellows segment 226e of the at least one flexible and extendable hollow tube 226 and two second stomach body securing component 244b along the second corrugated bellows segment 226f of the at least one flexible and extendable hollow tube 226. According to various embodiments, each of the two first stomach body securing component 244a and the two second stomach body securing component 244b may include a pair of fixing lugs 245 respectively extending sideways in opposite directions from the first corrugated bellows segment 226e of the at least one flexible and extendable hollow tube 226 and the second corrugated bellows segment 226f of the at least one flexible and extendable hollow tube 226.

In the following, results of studies and experiments conducted based on a prototype device according to the gastric restrictive device 100, 200 of the various embodiments are presented.

The prototype devices were three dimensional (3D) printed or built using the SOLIDWORKS CAD software and the POLYJET-OBJET500 machine with a flexible photopolymer, AGILUS30, which has superior tear-resistance (5-7 Kg/cm) and capable of withstanding repeated flexing and bending with tensile strength of 2.4-3.1 MPa. Although AGILUS30 is not the most ideal material for rapid prototyping and device validation, this rubber-like material is still able to simulate the look, feel and function of rubber-like products. According to various embodiments, the gastric restrictive device 100, 200 may be made with medical grade silicone, which is not exactly rubber-like. However, AGILUS30 did eventually provide good prototype performance. For the hard inner core (i.e. the first elongated rigid member 112, 212 and the second elongated rigid member 114, 214), VERO WHITE material was used. With a flexural strength of 75-110 MPa and tensile strength of 60-70 MPa, VERO WHITE, material is not as strong as metal but the fact that it can be jointly or integrally printed with AGILUS30 using the same machine made it a suitable choice for constructing the prototype. According to various embodiments, in the gastric restrictive device 100, 200, this hard core (i.e. the first elongated rigid member 112, 212 and the second elongated rigid member 114, 214) may be made of a bio-compatible metal, e.g. titanium alloy.

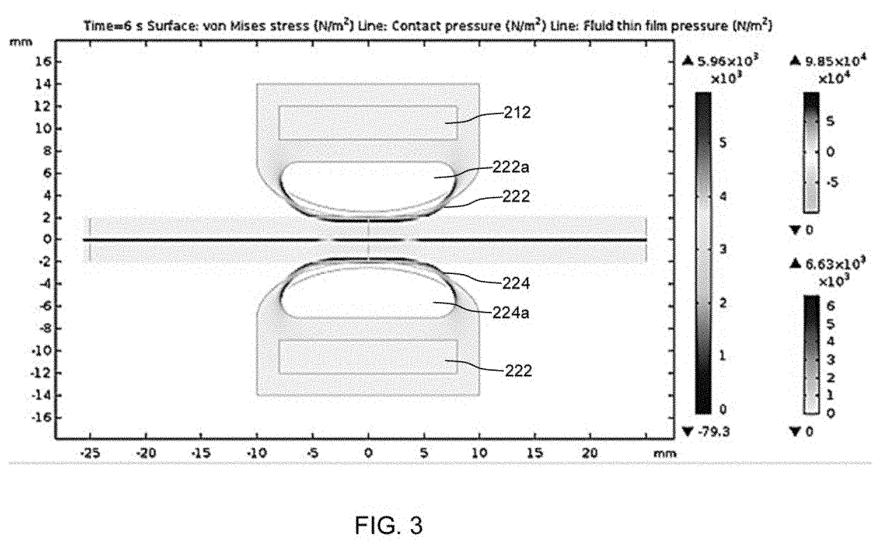

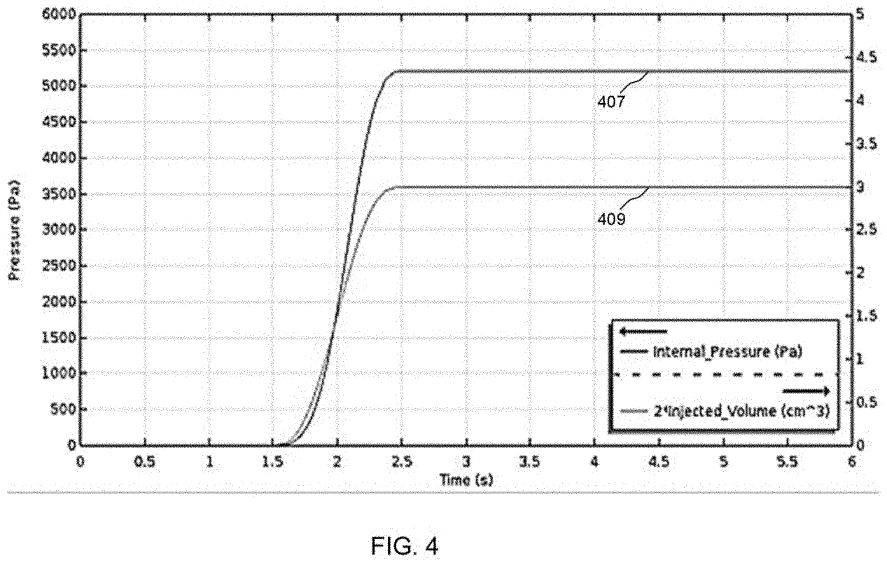

Simulation studies were conducted by COMSOL software with consideration for the AMERICAN SOCIETY OF MECHANICAL ENGINEERING (ASME) Verification & Validation (V&V40) standards. The contact pressure applied on the tissue and the sealing pressure between the two layers of the clipped tissues have been calculated when specified incompressible fluid (water-based solution) is injected into the prototype device. It is shown that the sealing pressure exceeds known reported stomach interior pressure. Simulation studies were also conducted for the more ideal material properties for the gastric restrictive device 100, 200, e.g. silicon polymer and metal core, which were not used in the 3D-printed prototype device. In addition, some of the advanced modelling features that have been implemented to simulate the actual physical behaviour of the gastric restrictive device 100, 200 include: compressing of internal air by injecting incompressible solution, e.g. nonlinear change in interior air pressure based on the air state equation; tissue-device mechanical contact using penalty method; nonlinear material behavior of the stomach soft tissue based on known characterization; and thin film flow and corresponding structural interaction with tissue. FIG. 3 shows an example illustration of the contact and sealing pressure obtained from the simulation studies. FIG. 4 shows an example illustration of the rise of sealing pressure 407 with injection of water-based solution 409 obtained from the simulation studies.

Benchtop experiments were carried out using fruit/vegetable pulp as food and zip-lock plastic bag as stomach with the prototype device. Half of the zip-lock bag was filled with pulp. The prototype device was mounted on the zip-lock bag and then pressurized by injecting water. Highly satisfactory sealing performance was demonstrated against the gravity in the benchtop experiments.

An isolated pig stomach study was also performed. An endoscopic probe was used to observe inside the stomach, which was air pressurized. The isolated pig stomach study was mainly meant to test for surgical feasibility and design fixation, including the length, suture point locations, etc.

The studies and experiments have shown the viability and feasibility of the gastric restrictive device 100, 200. Further, the studies and experiments have also shown that additive manufacturing especially with flexible materials has opened new windows for the rapid prototyping of the gastric restrictive device 100, 200, so as to achieve the optimal design with minimal cost and effort. Novel ways were explored for minimizing the post processing time, printing hollow parts and flexible one-way valve, to successfully complete the prototype of the new design. According to various embodiments, in future, additive manufacturing may become the preferred manufacturing method as the size of the gastric restrictive device 100, 200 may be customized for each patient.

The following pertains to various embodiments.

According to various embodiments, there is provided a gastric restrictive device. The gastric restrictive device may include two separate elongated rigid members. The gastric restrictive device may include a cushioning arrangement including at least one first elongated inflatable padding lined longitudinally along a first elongated rigid member of the two separate elongated rigid members, at least one second elongated inflatable padding lined longitudinally along a second elongated rigid member of the two separate elongated rigid members, and at least one flexible and extendable hollow tube fluidly interconnecting the at least one first elongated inflatable padding and the at least one second elongated inflatable padding. A first end of the at least one flexible and extendable hollow tube may be connected to an end of the at least one first elongated inflatable padding corresponding to a first longitudinal end of the first elongated rigid member, and a second end of the at least one flexible and extendable hollow tube may be connected to an end of the at least one second elongated inflatable padding corresponding to a first longitudinal end of the second elongated rigid member. The gastric restrictive device may include an interlocking arrangement including a first interlocking element at a second longitudinal end of the first elongated rigid member, and a second interlocking element at a second longitudinal end of the second elongated rigid member. The at least one flexible and extendable hollow tube of the cushioning arrangement may be bendable to fold the two separate elongated rigid members towards each other from a non-closed configuration into a closed configuration. In the non-closed configuration, the first interlocking element and the second interlocking element may be spaced apart from each other in a non-engageable disposition. In the closed configuration, the at least one first elongated inflatable padding and the at least one second elongated padding may be parallel and opposing each other and the first interlocking element and the second interlocking element may be engageable with each other to interlock in the closed configuration.

According to various embodiments, the gastric restrictive device described in the paragraph immediately above may optionally include that the at least one flexible and extendable hollow tube of the cushioning arrangement may include a tube with corrugated bellows.

According to various embodiments, the gastric restrictive device as described in any one of the two preceding paragraphs above may optionally include that the at least one flexible and extendable hollow tube may include a first corrugated bellows segment, a second corrugated bellows segment, and a smooth segment extending between the first corrugated bellows segment and the second corrugated bellows segment.

According to various embodiments, the gastric restrictive device as described in the paragraph immediately above may optionally include a branch tube extending from a side of the smooth segment of the at least one flexible and extendable hollow tube in a manner so as to be in fluid communication with the smooth segment of the at least one flexible and extendable hollow tube.

According to various embodiments, the gastric restrictive device as described in the paragraph immediately above may optionally include that the branch tube may include a one-way check valve for one-way fluid flow from the branch tube into the smooth segment of the at least one flexible and extendable hollow tube.

According to various embodiments, the gastric restrictive device as described in the paragraph immediately above may optionally include that the one-way check valve may be located at an end of the branch tube that is in fluid connection with the smooth segment of the at least one flexible and extendable hollow tube.

According to various embodiments, the gastric restrictive device as described in the paragraph immediately above may optionally include that an opposite end of the branch tube may be configured to be coupled to a fluid source for injecting a fluid via the branch tube through the one-way check valve into the at least one flexible and extendable hollow tube, and from the at least one flexible and extendable hollow tube into the at least one first elongated inflatable padding and the at least one second elongated inflatable padding respectively to inflate the at least one first elongated inflatable padding and the at least one second elongated inflatable padding.

According to various embodiments, the gastric restrictive device as described in any one of the seven preceding paragraphs above may optionally include that the first elongated rigid member with the at least one first elongated inflatable padding and the second elongated rigid member with the at least one second elongated inflatable padding may be cooperatively operable to clamp a stomach in the closed configuration so as to restrict the stomach in a reversible anatomically manner.

According to various embodiments, the gastric restrictive device as described in any one of the eight preceding paragraphs above may optionally include a securing arrangement having a stomach fundus securing component along the first elongated rigid member or the second elongated rigid member, and at least one stomach body securing component along the at least one flexible and extendable hollow tube of the cushioning arrangement and interposed between the first end and the second end of the at least one flexible and extendable hollow tube.

According to various embodiments, the gastric restrictive device as described in the paragraph immediately above may optionally include that the stomach fundus securing component may include an eyelet structure.

According to various embodiment, the gastric restrictive device as described in any one of the two preceding paragraphs above may optionally include that the at least one stomach body securing component may include a fixing lug extending sideways from the at least one flexible and extendable hollow tube of the cushioning arrangement.

According to various embodiments, the gastric restrictive device as described in the paragraph immediately above may optionally include that the at least one stomach body securing component may include a pair of fixing lugs extending sideways in opposite directions from a point along the at least one flexible and extendable hollow tube of the cushioning arrangement.

According to various embodiments, gastric restrictive device as described in any one of the four preceding paragraphs above may optionally include that, when the at least one flexible and extendable hollow tube may include a first corrugated bellows segment, a second corrugated bellows segment, and a smooth segment extending between the first corrugated bellows segment and the second corrugated bellows segment, the at least one stomach body securing component may be along the first corrugated bellows segment or the second corrugated bellows segment of the at least one flexible and extendable hollow tube of the cushioning arrangement.

According to various embodiments, the gastric restrictive device as described in the paragraph immediately above may optionally include that the securing arrangement may include a first stomach body securing component along the first corrugated bellows segment of the at least one flexible and extendable hollow tube and a second stomach body securing component along the second corrugated bellows segment of the at least one flexible and extendable hollow tube.

According to various embodiments, the gastric restrictive device as described in any one of the fourteen preceding paragraphs above may optionally include that that the first interlocking element of the interlocking arrangement may include an elongated insert member extending backward from the second longitudinal end of the first elongated rigid member in a manner so as to overhang above a portion of the at least one first elongated inflatable padding along the first elongated rigid member, and the second interlocking element of the interlocking arrangement may include a corresponding elongated hole extending longitudinally into the second elongated rigid member from the second longitudinal end of the second elongated rigid member.

According to various embodiments, the gastric restrictive device as described in the paragraph immediately above may optionally include that the elongated insert member of the first elongated rigid member may include at least one tooth along the elongated insert member, and the corresponding elongated hole of the second elongated rigid member may include at least a corresponding socket for receiving the at least one tooth of the elongated insert member when the elongated insert member of the first elongated rigid member is inserted into the corresponding elongated hole of the second elongated rigid member.

According to various embodiments, the gastric restrictive device as described in the paragraph immediately above may optionally include that the elongated insert member of the first elongated rigid member may include an elongated plate-like structure having two rows of ratchet-like teeth respectively along two opposite longitudinal sides of the elongated plate-like structure, and the corresponding elongated hole of the second elongated rigid member may include a rectangular cross-section having two rows of sockets respectively along two opposing longitudinal sidewalls of the corresponding elongated hole.

Various embodiments have provided a gastric restrictive device which may be as effective as sleeve gastrectomy but in a reversible manner, which does not involve surgically removing a large portion of the stomach. Further, the gastric restrictive device may be configured according to the anatomy of the stomach as well as the physiology of the stomach, such as the peristalsis movement of the stomach, to minimise or avoid damages or bruising of the stomach tissue. Accordingly, the gastric restrictive device provided an enhanced solution which may be effective and balanced for bariatric surgery.

While the invention has been particularly shown and described with reference to specific embodiments, it should be understood by those skilled in the art that various changes, modification, variation in form and detail may be made therein without departing from the scope of the invention as defined by the appended statements. The scope of the invention is thus indicated by the appended statements and all changes which come within the meaning and range of equivalency of the statements are therefore intended to be embraced.

1. A gastric restrictive device comprising:
two separate elongated rigid members;
a cushioning arrangement comprising
at least one first elongated inflatable padding lined longitudinally along a first elongated rigid member of the two separate elongated rigid members,
at least one second elongated inflatable padding lined longitudinally along a second elongated rigid member of the two separate elongated rigid members, and
at least one flexible and extendable hollow tube fluidly interconnecting the at least one first elongated inflatable padding and the at least one second elongated inflatable padding,
wherein a first end of the at least one flexible and extendable hollow tithe is connected to an end of the at least one first elongated inflatable padding corresponding to a first longitudinal end of the first elongated rigid member, and a second end of the at least one flexible and extendable hollow tube is connected to an end of the at least one second elongated inflatable padding corresponding to a first longitudinal end of the second elongated rigid member; and
an interlocking arrangement comprising
a first interlocking element at a second longitudinal end of the first elongated rigid member, and
a second interlocking element at a second longitudinal end of the second elongated rigid member,
wherein the at least one flexible and extendable hollow tube of the cushioning arrangement is bendable to fold the two separate elongated rigid members towards each other from a non-closed configuration, whereby the first interlocking element and the second interlocking element are spaced apart from each other in a non-engageable disposition, into a closed configuration with the at least one first elongated inflatable padding and the at least one second elongated padding parallel and opposing each other and with the first interlocking element and the second interlocking element engageable with each other to interlock in the closed configuration.

2. The device as claimed in claim 1, wherein the at least one flexible and extendable hollow tube of the cushioning arrangement comprises a tube with corrugated bellows.

3. The device as claimed in claim 1, wherein the at least one flexible and extendable hollow tube comprises a first corrugated bellows segment, a second corrugated bellows segment, and a smooth segment extending between the first corrugated bellows segment and the second corrugated bellows segment.

4. The device as claimed in claim 3, further comprising a branch tube extending from a side of the smooth segment of the at least one flexible and extendable hollow tube in a manner so as to be in fluid communication with the smooth segment of the at least one flexible and extendable hollow tube.

5. The device as claimed in claim 4, wherein the branch tube comprises a one-way check valve for one-way fluid flow from the branch tube into the smooth segment of the at least one flexible and extendable hollow tube.

6. The device as claimed in claim 5, wherein the one-way check valve is located at an end of the branch tube that is in fluid connection with the smooth segment of the at least one flexible and extendable hollow tube.

7. The device as claimed in claim 6, wherein an opposite end of the branch tube is configured to be coupled to a fluid source for injecting a fluid via the branch tube through the one-way check valve into the at least one flexible and extendable hollow tube, and from the at least one flexible and extendable hollow tube into the at least one first elongated inflatable padding and the at least one second elongated inflatable padding respectively to inflate the at least one first elongated inflatable padding and the at least one second elongated inflatable padding.

8. The device as claimed in claim 1, wherein the first elongated rigid member with the at least one first elongated inflatable padding and the second elongated rigid member with the at least one second elongated inflatable padding are cooperatively operable to clamp a stomach in the closed configuration so as to restrict the stomach in a reversible anatomically manner.

9. The device as claimed in claim 1, further comprising a securing arrangement having
a stomach fundus securing component along the first elongated rigid member or the second elongated rigid member, and
at least one stomach body securing component along the at least one flexible and extendable hollow tube of the cushioning arrangement and interposed between the first end and the second end of the at least one flexible and extendable hollow tube.

10. The device as claimed in claim 9, wherein the stomach fundus securing component comprises an eyelet structure.

11. The device as claimed in claim 9, wherein the at least one stomach body securing component comprises a fixing lug extending sideways from the at least one flexible and extendable hollow tube of the cushioning arrangement.

12. The device as claimed in claim 11, wherein the at least one stomach body securing component comprises a pair of fixing lugs extending sideways in opposite directions from a point along the at least one flexible and extendable hollow tube of the cushioning arrangement.

13. The device as claimed in claim 9, wherein the at least one flexible and extendable hollow tube comprises a first corrugated bellows segment, a second corrugated bellows segment, and a smooth segment, and a smooth segment extending between the first corrugated bellows segment and the second corrugated bellows segment, further wherein the at least one stomach body securing component is along the first corrugated bellows segment or the second corrugated bellows segment of the at least one flexible and extendable hollow tube of the cushioning arrangement.

14. The device as claimed in claim 13, wherein the securing arrangement comprises a first stomach body securing component along the first corrugated bellows segment of the at least one flexible and extendable hollow tube and a second stomach body securing component along the second corrugated bellows segment of the at least one flexible and extendable hollow tube.

15. The device as claimed in claim 1,
wherein the first interlocking element of the interlocking arrangement comprises an elongated insert member extending backward from the second longitudinal end of the first elongated rigid member in a manner so as to overhang above a portion of the at least one first elongated inflatable padding along the first elongated rigid member, and
wherein the second interlocking element of the interlocking arrangement comprises a corresponding elongated hole extending longitudinally into the second elongated rigid member from the second longitudinal end of the second elongated rigid member.

16. The device as claimed in claim 15,
wherein the elongated insert member of the first elongated rigid member comprises at least one tooth along the elongated insert member, and
wherein the corresponding elongated hole of the second elongated rigid member comprises at least a corresponding socket for receiving the at least one tooth of the elongated insert member when the elongated insert member of the first elongated rigid member is inserted into the corresponding elongated hole of the second elongated rigid member.

17. The device as claimed in claim 16,
wherein the elongated insert member of the first elongated rigid member comprises an elongated plate-like structure having two rows of ratchet-like teeth respectively along two opposite longitudinal sides of the elongated plate-like structure, and
wherein the corresponding elongated hole of the second elongated rigid member comprises a rectangular cross-section having two rows of sockets respectively along two opposing longitudinal sidewalls of the corresponding elongated hole.
